(19) United States
(12) Patent Application Publication
(10) Pub. No.: US 2025/0109445 A1
(43) Pub. Date: Apr. 3, 2025

(54) DNA METHYLATION BIOMARKERS FOR EARLY DETECTION OF CERVICAL CANCER

(71) Applicant: HKG EPITHERAPEUTICS LIMITED, Kowloon (HK)

(72) Inventors: Moshe Szyf, Cote St Luc (CA); David Cheishvili, Pierrefonds (CA); Eduardo Franco, Montreal (CA); Mariam El-Zein, Montreal (CA)

(21) Appl. No.: 18/931,575

(22) Filed: Oct. 30, 2024

Related U.S. Application Data
(62) Division of application No. 17/299,132, filed on Jun. 2, 2021.
(60) Provisional application No. 62/774,994, filed on Dec. 4, 2018.

(51) Int. Cl. C12Q 1/6886; G16B 20/20

(52) U.S. Cl. CPC C12Q 1/6886; G16B 20/20; C12Q 2600/154

(57) ABSTRACT

The present invention discloses an in vitro method for obtaining DNA methylation biomarkers as exquisite DNA methylation positions in the human genome (i.e., CGIDs) that predict cervical cancer especially at as yet inaccessible early stages by examining progression of “categorical” DNA methylation alterations in three stages of premalignant lesions (cervical intraepithelial neoplasia (CIN)), progressing from CIN1 to CIN3. The present invention discloses combinations of CGIDs for detecting with high specificity and sensitivity cervical cancer by measuring their DNA methylation status and deriving a “methylation score”, which is useful as a biomarker for cervical cancer. Also disclosed are kits for predicting cervical cancer using such CGIDs using multiplexed next generation sequencing methylation assays, pyrosequencing assays and methylation specific PCR. The DNA methylation markers (CGIDs) described in the present invention are useful for cervical screening and early detection of cervical cancer by any person skilled in the art to detect cervical cancer.

### CROSS-REFERENCE TO RELATED APPLICATIONS

This application is a divisional of application Ser. No. 17/299,132 filed on Jun. 2, 2021 which claims priority under Section 119(e) from U.S. Provisional Application Ser. No. 62/774,994, filed Dec. 4, 2018, entitled “DNA METHYLATION MARKERS FOR EARLY DETECTION OF CERVICAL CANCER” the contents of each which are incorporated herein by reference.

### SEQUENCE LISTING

The instant application contains a Sequence Listing XML which has been submitted electronically in XML format and is hereby incorporated by reference in its entirety. Said XML copy, created on Dec. 19, 2024, is named TPC53811 Seq List_ST26.xml and is 129,092 bytes in size.

### TECHNICAL FIELD

The present invention relates generally to DNA methylation signatures in human DNA, particularly in the field of molecular diagnostics. More specifically, the present invention is DNA methylation biomarkers in the form of a panel, individual as well as combination of polygenic DNA methylation biomarkers for early detection as well as screening of cervical cancer, and their use as a diagnostic kit for early and accurate detection of cervical cancer.

### BACKGROUND OF THE INVENTION

Cancer has become a major killer of humans. Early detection of cancer can significantly improve cure rates and reduce the horrific personal and financial cost to the patients, their families and the health care system. At the same time, screening of healthy individuals to assess for pre-cancerous stage biomarker expression and its alterations is useful in a population-wide screening methodology and helpful in identifying risk-prone, cancer susceptible, healthy individuals. Cervical cancer is no exception. Screening can identify cancer at an early stage-before it can cause symptoms. If cervical cancer is caught at its earliest stage, the chance of survival is about 93% and it goes down to 15% for the latest stages https://www.cancer.org/cancer/cervical-cancer/detection-diagnosis-staging/survival.html. Current screening methods include Pap smears, liquid based cytology, HPV testing and visual inspection, however a robust highly accurate and sensitive method for early detection of cervical cancer is lacking.

Biomarkers constitute one of the most important fields in cancer diagnosis. Cancer biomarkers are especially useful for early detection or diagnosis of the disease. Biomarkers can be used to screen patients, for classifying the different stages or grades of cancers and to predict prognosis and resistance to therapy.

The well-established discovery of human papillomavirus (HPV) as the causative agent of cervical neoplasia has revolutionized the prevention and management modalities of this gynecological disease from a secondary (molecular HPV testing) standpoint (1). Knowledge of the HPV genotype is indeed helpful in clinical prediction, as HPVs 16 and/or 18 types are associated with greater risks of lesion progression than other carcinogenic types. However, persistent infection with carcinogenic HPV genotypes is the necessary precursor and driver in cervical carcinogenesis. The latter represents a stepwise progression from premalignant stages (cervical intraepithelial neoplasia, CIN) to invasive cervical cancer. Low-grade CIN (CIN1) is eminently reversible whereas high-grade CIN of grades 2 and 3 (i.e., CIN2 and CIN3, respectively) have a non-negligible risk of progressing to invasion, i.e., cervical cancer. This is particularly true for CIN3.

Managing women with CIN pathologies in the clinic continues to pose a significant dilemma for the gynecologists, as aggressive ablative or excisional treatment may cause immediate complications or increase the risk of miscarriage or premature delivery later in life, when the female patient decides to become pregnant. Recent evidence suggests that epigenetic changes in specific genes may mediate or predict carcinogenic progression. A cancer early detection biomarker can categorically differentiate rare cells with lesions at asymptomatic and precancerous stages due to remarkable changes which include biochemical changes at the epigenetic levels. These epigenetic changes as biomarkers are quite often produced in abnormally large numbers in the cancerous tissues and often preclude manifestation of the disease itself. To identify molecular changes setting-in much before the disease initiation and progression, development of molecular biomarkers is extremely important. One such epigenetic biomarkers DNA methylation levels of certain CpG sites in viral and host genes were shown to increase with the severity of the underlying cervical lesions (2-7).

Among the most-studied and targeted host genes with epigenetic changes associated with cervical cancer and its precursors are cell adhesion molecule 1 (CADM1); death associated protein kinase 1 (DAPK1); myelin and lymphocyte, T cell differentiation protein (MAL); paired box 1 (PAX1); telomerase reverse transcriptase (TERT); erythrocyte membrane protein band 4.1-like 3 (EPB41L3), Ras association domain family member 1 (RASSF1); SRY-box 1 (SOX1); cadherin 1 (CDH1); LIM homeobox transcription factor 1 alpha (LMX); cyclin A1 (CCNA1); family with sequence similarity 19 member A4, C-C motif chemokine-like (FAM19A4); and retinoic acid receptor beta (RARB) 8. Single (9) methylation markers were investigated in addition to those that included two (i.e., CADM1 and MAL(3,4,10); MAL and miR124-2 (11-14), three (i.e., CADM1, MAL, and miR124-2) (13,15), four (i.e., JAM3, EPB41L3, TERT, and C13ORF18) (16,17), and five (i.e., PAX1, DAPK1, RARβ, WIF1, and SLIT2) (14) marker panels as well as panels involving various combinations of SOX1, PAX1, LMX1A and NKX6-1 markers to attain sufficiently high sensitivities for advanced lesions (18).

However, only one previous study, using a genome-wide methylation approach identified three methylation panels (JAM3/ANKRD18CP, C13ORF18/JAM3/ANKRD18CP, and JAM3/GFRA1/ANKRD18CP) with the highest combined diagnostic accuracy for the detection of CIN2+ in cervical samples; and the sensitivities were reported to be 72%, 74% and 73%, respectively, with corresponding specificities of 79%, 76% and 77% (2). Accordingly, there is a need for improved methods of identification of DNA methylation biomarkers, panel of DNA methylation biomarkers associated with early detection and risk prediction of cervical cancer as well as kits based on such biomarkers for population-wide screening of apparently healthy women for early detection and susceptibility for cervical cancer and for risk assessments of women with pre-cancerous pathologies.

The present invention provides a solution to the problem associated with lack of early detection markers of cervical cancer by using DNA methylation biomarkers as singular, combination as well as panel-based biomarkers, since there lacks a single or combined methylation marker that has the appropriate diagnostic performance for risk prediction of cervical cancer at an early stage at present. The present invention discloses a method for obtaining early biomarkers of progression of premalignant lesion to cervical cancer that could be used for general screening in non-symptomatic as well as women displaying the CIN1 to CIN3 pathologies.

### OBJECTIVES OF THE INVENTION

The main objective of the present invention relates to biomarkers for early detection and diagnosis of human cervical cancer.

Further objective of the present invention relates to an in vitro method disclosed herein called “analysis of progressive DNA methylation alterations (APDMA)” that involves steps of examining genome wide profiles of DNA methylation of specimens from women with different CIN grade pathologies (CIN1 to CIN3) as compared to healthy control specimens from women for obtaining CGIDs as DNA methylation biomarkers that predict when combined, using a linear regression model disclosed here, cervical cancer with >95% sensitivity and specificity in publicly available methylation profiles of cervical cancer.

Another objective of the present invention relates to molecular biomarkers as indicators of population-wide screening of women for early detection of cervical cancer as well as for risk assessments of women with CIN1 to CIN3 pathologies.

Yet another objective of the present invention relates to a chip/array useful for early detection and diagnosis of cervical cancer.

Still another objective of the present invention is to provide a cheaper, accurate, robust, highly sensitive and specific, and high throughput diagnostic kit for accurate early diagnosis of human cervical cancer usable by any person skilled in the art.

### SUMMARY OF THE INVENTION

Accordingly, the present invention provides methods and materials useful to examine DNA methylation alterations and relates to DNA methylation CGID biomarkers for early detection and diagnosis of human cervical cancer, wherein the progression of premalignant cervical lesions (cervical intraepithelial neoplasia, CIN grades 1 to 3) correlates with increased frequency of DNA methylation at CG positions in the human genome in the form of Illumina probe ID or DNA methylation number or CG identifiers (CGIDs) which are obtained using the presently disclosed in vitro method of “analysis of progressive DNA methylation alterations” (APDMA) as disclosed herein. As discussed in detail below, typically these biomarkers are based on variables that lend themselves to predicting risk of women with CIN1 to CIN3 pathologies as well as in population-wide screens for developing cervical cancer, and in turn useful as early detection and diagnosis biomarkers. The present disclosure provides that the said CGID biomarker positions are almost uniformly methylated in cervical cancer and almost uniformly unmethylated in normal cervical specimens. The present invention thus, discloses the said set of “categorically” distinct DNA methylation profiles that create a binary differentiation between cervical cancer and nonmalignant tissues in the form of DNA methylation at these CGID sites, whereby these sites are only methylated in cervical cancer and fully unmethylated in nonmalignant tissue. Moreover, as disclosed herein these biomarker sites show an increasing frequency of DNA methylation with the progression of premalignant cervical lesions going from CIN1 to CIN3. Thus, the present invention provides an early detection and diagnosis in vitro method using targeted amplification of the said CGID biomarkers and deep next generation bisulfite sequencing to detect even a few molecules of cervical cancer cells or even the cells from premalignant lesions on the trajectory to becoming cervical cancer on the background of mostly normal cervical cell profile. The present invention is thus useful for as yet inaccessible early detection of cervical cancer cells on a high background of nonmalignant tissue particularly using cervical specimens such as pap smears as an easy and user-friendly method of early detection usable by any person of skill in the art.

An embodiment of the present invention relates to an in vitro method for obtaining highly predictive sites for cervical cancer for early detection even at the asymptomatic and premalignant stages called the “analysis of progressive DNA methylation alterations (APDMA) method” using different sources of genome wide DNA methylation data derived by next generation sequencing, including, MeDIP arrays, MeDIP sequencing etc., obtained in the form of DNA methylation CGID biomarker signatures. The present invention provides a combination of “categorical” CGID biomarkers for detection of cervical cancer in a discovery set of genome wide data from specimens of progressive premalignant lesions going from CIN1 to CIN3.

Previous analyses before the present invention using classic “case-control” design and logistic regressions had revealed DNA methylation CGID biomarkers that detect cancer at lower sensitivity and specificity. Thus, another embodiment of the present invention relates to a computer-implemented method for obtaining candidate DNA methylation biomarkers for early detection for cervical cancer diagnosis, termed the APDMA method which reveals the earliest methylation profiles of cancer that are primary and essential for the cancer state and are thus present in all cervical cancer specimens tested in the disclosure herein.

An embodiment of the present invention discloses an in vitro method that accurately detects cervical cancer by measuring DNA methylation in a polygenic set of CGID biomarkers in hundreds of people concurrently, by sequential amplification with target specific primers followed by barcoding primers and multiplexed sequencing in a single next generation Miseq sequencing reaction, data extraction and quantification of methylation.

An embodiment of the present invention discloses an in vitro method of measurement of methylation of said DNA methylation CGID biomarkers using pyrosequencing assays or methylation specific PCR. The present invention discloses the calculation of a polygenic weighted methylation score that predicts cervical cancer.

An embodiment of the present invention discloses a panel of DNA methylation biomarkers for screening, diagnosis, early detection and prediction of cervical cancer in a sample of DNA isolated from a specimen from a woman including women with no other clinical evidence for cervical cancer from cervical specimens.

An embodiment of the present invention discloses a panel of DNA methylation biomarkers in form of a chip for screening, diagnosis, early detection and prediction of cervical cancer in a sample of DNA isolated from a specimen from a woman including women with no other clinical evidence for cervical cancer from cervical specimens.

An embodiment of the present invention discloses an in vitro non-invasive method using the panel of DNA methylation biomarkers for screening, diagnosis, early detection and prediction of cervical cancer in a sample of DNA isolated from a specimen from a woman including women with no other clinical evidence for cervical cancer from cervical specimens.

An embodiment of the present invention discloses a use of the DNA methylation biomarkers as disclosed herein for screening, diagnosis, early detection and prediction of cervical cancer in a sample of DNA isolated from a specimen from a woman including women with no other clinical evidence for cervical cancer from cervical specimens.

The present invention provides with robust DNA methylation biomarkers identified using CGID positions in the human genome that provide a highly accurate, specific and sensitive assessment of risk that can guide early intervention and treatment of cervical cancer even in women at asymptomatic and precancerous stages. The present invention provides an easy yet efficient method that could be used by any person skilled in the art to detect cervical cancer. The present invention relates to the use of the disclosed DNA methylation CGID biomarkers described herein for population-wide screening of healthy women for cervical cancer as well as for monitoring and assessing cancer risk in women with HPV infection and CIN premalignant lesions. The present invention demonstrates the utility of the disclosed DNA methylation biomarkers in detecting cervical cancer in CIN samples using a polygenic score based on the DNA methylation measurement methods disclosed herein. The present invention also discloses the utility of the disclosed method for obtaining “polygenic” categorical DNA methylation CGID biomarkers for cervical cancer using any method available to people skilled in the art for genome wide bisulfite sequencing such as next generation bisulfite sequencing, MeDip sequencing, ion torrent sequencing, Illumina 450 K arrays and Epic microarrays etc., followed by the presently disclosed APDMA method as disclosed herein, for discovering specific and sensitive markers useful for early and very early detection of cervical cancer because of their categorical difference in DNA methylation profile between healthy controls and cervical cancer specimens with a gradation of increasing frequency when progressing from specimens from CIN1 to CIN3 precancerous stages.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF DRAWINGS

FIG. 1. Roadmap for developing analysis of progressive DNA methylation alterations (APDMA) method for obtaining the early detection DNA methylation biomarkers. The roadmap depicts the analytical procedure for developing the APDMA method based on the DNA methylation profile using Illumina assay probe identification (CGID) which categorically differentiate the normal profile of cervical specimens from the DNA methylation profile in the cervical cancer specimens for obtaining “categorical” DNA methylation CGID biomarkers for early detection, diagnosis as well as screening for cervical cancer. In step 1, DNA methylation measurements are obtained from cervical specimens of premalignant lesions CIN1 to CIN3 stages compared to healthy control specimens, said DNA methylation measurements are obtained either by performing Illumina Beadchip 450K or 850K assay of DNA extracted from the specimens or by performing DNA pyrosequencing of DNA extracted from sample or by mass spectrometry based (Epityper™), or by PCR based methylation assays and targeted amplification of a region spanning the target CGIDs disclosed here from bisulfite converted DNA followed by barcoding in a second set of amplification and indexed multiplexed sequencing on an Illumina next generation sequencer. In step 2, statistical analysis method is performed on the DNA methylation measurements of step 1, wherein the statistical analysis includes Receiver operating characteristics (ROC) assays, hierarchical clustering analysis assays, or neural networ c k analysis. In step 3, the presently developed and disclosed “analysis of progressive DNA methylation alterations” (APDMA) method is performed to identify CGID positions whose methylation levels are an early predictor or biomarker of cervical cancer. In step 4, the present disclosure further narrows and shortlists the polygenic DNA methylation CGID combinations as a biomarker set of 16 CGIDs. The method allows for obtaining “categorical” rather than quantitative differences in methylation profiles between normal and cervical cancer cells that in turn allows for early detection because of the characteristic switch in DNA methylation profile at the select CGIDs that provide as the DNA methylation biomarkers for early detection, diagnosis and screening for cervical cancer. These serve as a panel of candidate CGID biomarkers for early detection of cervical cancer in women, particularly those who are asymptomatic or with premalignant lesions.

FIG. 2. Method to obtain sites whose frequency of methylation increases progressively through premalignant CIN stages. DNA prepared from cervical specimens from specimens from CIN1, CIN2, and CIN3 histology individuals; and non-transformed, healthy controls was subjected to genome wide DNA methylation analysis on Illumina Epic Arrays. The level of methylation of 7715 CGIDs correlated significantly (q>0.05) with progression of premalignant CIN stages from CIN1 to CIN3. A. IGV browser view of the difference in methylation of these sites from control cervical specimens across the genome. Top track shows positions of chromosomes. Second track shows the position of Refseq genes across the genome. The following tracks (ΔCIN1−Ctrl, ΔCIN2−Ctrl, ΔCIN3−Ctrl) show the difference in average methylation between each of the CIN stages and controls. Progressive hypermethylation through the stages is observed.

FIG. 3. Sites derived by the APDMA method are categorically different between normal cervical specimens and cervical cancer. A. Heatmap showing that 79 top CGIDs whose frequency of methylation increases during progression of cervical premalignant phases detect cervical cancer using DNA methylation data from 270 patients (GSE68339). The CGIDs exhibit a categorically different methylation profile between cancer and normal cervix. They are totally unmethylated in normal tissue and heavily methylated in cancer tissue. B. Average methylation for each of the groups normal, premalignant stages and cervical cancer (CIN1 to CIN3) (blue refers to 0% methylation and dark red refers to 100% methylation).

FIG. 4. Specificity and specificity of a bi-genic DNA methylation score discovered using the APDMA method for detecting cervical cancer DNA in an independent cohort. A. Effect size calculation, penalized regression and multivariable linear regression short listed a subset of two CGIDs and a linear regression equation for prediction of cervical cancer was computed. B. A threshold for cancer detection was calculated by ROC. C. Using this threshold, the sensitivity and specificity of this combined set of markers is 1 and the AUC is 1.

FIG. 5. Cancer methylation scores in individual specimens from control CIN 1 to CIN3 and cervical cancer patients. A. Methylation scores (cervical cancer prediction) calculated using the equation presented in FIG. 4A for each of the individual specimens from controls, CIN1 to CIN3 and cervical cancer showing increase in methylation scores in advanced premalignant lesions. B. Scatter plot showing average methylation score for the control, premalignant and cancer groups.

FIG. 6. Correlation between bi-genic methylation score and progression from control through premalignant stages to cervical cancer. Cervical cancer samples are from GSE68339 CIN1 to CIN3 are from the McGill cohort described in this application (assigned Spearman rank: control: 0, CIN1 to CIN3: 1-3, cervical cancer: 4).

FIG. 7. Validation of methylation marker for cervical cancer using DNA methylation data from TCGA (n=312). Since data for cervical for only one CGIDs (cg13944175) was available in TCGA, we calculated the methylation score for cervical cancer using a linear regression equation with DNA methylation data for only CGID cg13944175. A Pearson correlation was calculated between stage of progression to cancer and the methylation score (see statistics in A and correlation chart in B). CIN1 to CIN 3 are from the McGill cohort described in this application. Assigned scales: Control: 0, CIN1 to CIN 3:1-3, Cervical cancer: 4.

FIG. 8. Utility of the present invention: Prediction of cervical cancer in CIN1 to CIN3 specimens. Not all CIN-1-3 patient develop cervical cancer though a higher fraction of CIN3 patients than CIN1 patients do. The present invention tested whether the methylation score developed in FIG. 3 be used to identify individual patients who exhibit a cervical cancer methylation score as a demonstration of the utility of the present invention. A. The X axis lines up individual patients, groups are indicated by the lines under the X axis. The Y axis indicates prediction of cancer (1) and no cancer call (0). B. Number of individuals with cancer prediction in each group. Prediction of cancer increases from CIN1 to CIN3 as expected.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All illustrations of the drawings are for the purpose of describing selected versions of the present invention and are not intended to limit the scope of the present invention.

All publications mentioned herein are incorporated herein by reference to disclose and describe aspects, methods and/or materials in connection with the cited publications.

DNA methylation refers to chemical modifications of the DNA molecule. Technological platforms such as the Illumina Infinium microarray or DNA sequencing-based methods have been found to lead to highly robust and reproducible measurements of the DNA methylation levels of a person. There are more than 28 million CpG loci in the human genome. Consequently, certain loci are given unique identifiers such as those found in the Illumina CpG loci database (see, e.g. Technical Note: Epigenetics, CpG Loci Identification ILLUMINA Inc. 2010). These CG locus designation identifiers are used herein.

### Definitions

As used herein, the term “CG” refers to a di-nucleotide sequence in DNA containing cytosine and guanosine bases. These di-nucleotide sequences could become methylated in the DNA from humans as well as other animals. The CGID reveals its position in the human genome as defined by the Illumina 450K manifest or Illumina EPIC manifest (the annotation of the CGs listed herein is publicly available at https://bioconductor.org/packages/release/data/annotation/html/IlluminaHumanMethylation4 50k.db.html or https://bioconductor.org/packages/release/data/annotation/html/IlluminaHumanMethylationE PICmanifest.html and installed as an R package IlluminaHumanMethylation450k.db (R package version 2.0.9.) or IlluminaHumanMethylationEPICmanifest (R package version 0.3.0).

As used herein, the term “beta-value” refers to computation of methylation level at a CGID position derived by normalization and quantification of Illumina 450K or EPIC arrays using the ratio of intensities between methylated and unmethylated probes and the formula: beta value=methylated C intensity/(methylated C intensity+unmethylated C intensity) between 0 and 1 with 0 being fully unmethylated and 1 being fully methylated.

As used herein, the term “penalized regression” refers to a statistical method aimed at identifying the smallest number of predictors required to predict an outcome out of a larger list of biomarkers as implemented for example in the R statistical package “penalized” as described in Goeman, J. J., L1 penalized estimation in the Cox proportional hazards model. Biometrical Journal 52(1), 70-84.

As used herein, the term “clustering” refers to the grouping of a set of objects in such a way that objects in the same group (called a cluster) are more similar (in some sense or another) to each other than to those in other groups (clusters).

As used herein, the term “Hierarchical clustering” refers to a statistical method that builds a hierarchy of “clusters” based on how similar (close) or dissimilar (distant) are the clusters from each other as described for example in Kaufman, L.; Rousseeuw, P. J. (1990). Finding Groups in Data: An Introduction to Cluster Analysis (1 ed.). New York: John Wiley. ISBN 0-471-87876-6.

As used herein, the term “Receiver operating characteristics (ROC) assay” refers to a statistical method that creates a graphical plot that illustrates the performance of a predictor. The true positive rate of prediction is plotted against the false positive rate at various threshold settings for the predictor (i.e. different % of methylation) as described for example in Hanley, James A.; McNeil, Barbara J. (1982). “The Meaning and Use of the Area under a Receiver Operating Characteristic (ROC) Curve”. Radiology 143(1):29-36.

As used herein, the term “multivariable or polygenic linear regression” refers to a statistical method that estimates the relationship between multiple “independent variables” or “predictors” such as percentage of methylation in multiple CGIDs, and a “dependent variable” such as cancer. This method determines the “weight” or coefficient of each CGIDs in predicting the “outcome” (dependent variable such as cancer) when several “independent variables” such as CGIDs are included in the model.

As used herein, the term “epigenetic” means relating to, being, or involving a chemical modification of the DNA molecule. Epigenetic factors include the addition or removal of a methyl group which results in changes of the DNA methylation levels. Novel molecular biomarkers of early detection or diagnosis or prediction of cervical cancer that observe methylation patterns in genomic DNA, such as those disclosed here as CGID based biomarkers allow one to prognosticate cervical cancer risk and susceptibility even at very early stages where the women are asymptomatic or at premalignant stages progressing from CIN1 to CIN3, and would be useful in the clinic, to epidemiologists, medical professionals, and are the present disclosure is made such that it be accessible and usable by any person skilled in the art. Exclusively clinical biomarkers such as pap smears, histological identification have a long and successful history in cervical cancer diagnosis however, they are marred by vast degrees of variability and being incapable for use in early detection of cervical cancer. By contrast, molecular biomarkers such as epigenetic markers in form of DNA methylation biomarkers have as yet been rarely used.

As used herein, the term “DNA methylation biomarker” refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The presently disclosed method posits that the frequency of cells that display a cervical cancer DNA methylation profile increases with the progression from CIN1 to CIN3 pathologies and that these methylation profiles are characteristic of earliest cervical cancer. Second, since cells that convert to cancer are rare in early premalignancy, the DNA methylation profile should be categorically different than the normal profile of cervical cells so as to be detected on a background of mostly nonmalignant cells at the earliest of stages. Third, these DNA methylation profiles should be present in all fully developed cervical cancer specimens if they are primary and critical characteristic of cervical cancer. Considering the aforementioned three prerequisites, the presently disclosed an in vitro method termed “analysis of progressive DNA methylation alterations (APDMA)” involves steps of examining genome wide profiles of DNA methylation of specimens isolated and obtained from women with different CIN grade pathologies (CIN1 to CIN3) compared with healthy, non-transformed, healthy control cervical specimens after well-characterized HPV genotyping, using Infinium Methylation EPIC arrays. The present invention discloses an in vitro method for obtaining Illumina probe ID or DNA methylation number or CG identifiers (CGIDs) as DNA methylation biomarkers that predict when combined, using a linear regression model disclosed here, cervical cancer with >95% sensitivity and specificity in publicly available methylation profiles of cervical cancer. The present invention also provides a panel of DNA methylation biomarkers for screening and early detection of cervical cancer, wherein the panel comprises of CGIDs having sequences selected from the group consisting of SEQ ID NO:1 to 79 as listed in Table 1 as well as shortlisted subsets thereof as listed in Table 2 and Table 3, respectively, as disclosed herein. The present invention further provides with two CGIDs as minimally sufficient to detect cervical cancer in publicly available DNA methylation data with sensitivity and specificity that approaches 1. The present invention also discloses kits for in vitro measuring the DNA methylation biomarkers as the DNA methylation levels of the disclosed CGIDs in DNA isolated from cervical specimens to be used for population-wide screening of women for early detection of cervical cancer as well as for risk assessments of women with CIN1 to CIN3 pathologies.

The invention disclosed herein has a number of embodiments. In an embodiment, the present invention provides polygenic DNA methylation CGID biomarkers of cervical cancer in cervical smears for early detection of cervical cancer, said polygenic DNA methylation biomarkers panel is derived using “analysis of progressive DNA methylation alterations (APDMA) method” disclosed in the present invention on genome wide DNA methylation derived by mapping methods, such as Illumina 450K or 850K arrays, genome wide bisulfite sequencing using a variety of next generation sequencing platforms, methylated DNA Immunoprecipitation (MeDIP) sequencing or hybridization with oligonucleotide arrays.

In an embodiment, the present invention provides the method for obtaining DNA methylation biomarkers for detecting cervical cancer, comprising the step of performing statistical analysis and the “analysis of progressive DNA methylation alterations (APDMA)” method disclosed in the present invention on DNA methylation measurements obtained from cervical specimens of premalignant lesions CIN1 to CIN3.

In an embodiment, the presently disclosed method comprises of performing statistical analysis and the “analysis of progressive DNA methylation alterations (APDMA)” method on DNA methylation measurements obtained from cervical specimens, said DNA methylation measurements are obtained by performing Illumina Beadchip 450K or 850K assay of the DNA extracted from the specimens. In another embodiment, said DNA methylation measurements are obtained by performing DNA pyrosequencing of DNA extracted from sample or by mass spectrometry based (Epityper™), or by PCR based methylation assays and targeted amplification of a region spanning the target CGIDs disclosed here from bisulfite converted DNA followed by barcoding in a second set of amplification and indexed multiplexed sequencing on an Illumina next generation sequencer. In a further embodiment, said statistical analysis includes Receiver operating characteristics (ROC) assays. In yet another embodiment, said statistical analysis includes hierarchical clustering analysis assays. In an additional embodiment, said statistical analysis includes neural network analysis.

In an embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers.

In another embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said measuring DNA methylation is performed using methods comprising, Illumina 27K, 450K or 850K arrays, genome wide bisulfite sequencing on platforms including, HiSeq, MiniSeq, MiSeq or NextSeq sequencers, torrent sequencing, methylated DNA Immunoprecipitation (MeDIP) sequencing, hybridization with oligonucleotide arrays, DNA pyrosequencing, mass spectrometry based (Epityper™) or PCR based methylation assays.

In yet another embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said statistical analysis on the DNA methylation measurement includes Pearson correlation, Receiver operating characteristics (ROC) assays, and hierarchical clustering analysis.

In a further embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said cervical cancer premalignant stage progression comprises cervical intraepithelial neoplasia lesions at stages CIN1, CIN2 and CIN3.

In an alternate embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said CGIDs based on frequency of their DNA methylation correlating with cervical cancer premalignant stage progression are selected from a group of CGIDs as set forth in SEQ ID NO:1 to SEQ ID NO:79. In a supplemental embodiment of the present invention, the 79 CGID sites are useful alone or in combination as early predictors of cervical cancer are delineated as DNA methylation biomarkers for early detection of cervical cancer.

### Table 1: Selected 79 Polynucleotides having CG Methylation Sites (CGIDs) Useful in Embodiments of the Present Invention

The 79 CGID biomarkers discussed herein are found in Table 1 that is included with this application. The said biomarkers were shortlisted as progressively methylated CGIDs with an average increase in methylation of 10% or decrease of more than 10% during transition from CIN1 to CIN3 stages and with a background methylation in normal cells (less than 10%) using the assumptions of the APDMA method as disclosed herein. The Illumina method takes advantage of sequences flanking a CG locus to generate a unique CG locus cluster ID with a similar strategy as NCBI's refSNP IDs (rs #) in dbSNP.

| SEQ ID NO | Probe | Probe with CpG marked |
|---|---|---|
| SEQ ID NO: 1 | cg08272731 | GAAGGAGGCTGCGCGCCAGCCCGCCCGCGGCGCCCGGGCTCAGGCGCC |
| | | GTGACGGCTGCA[CG]CGCTGCCCCGCACTCTGAGGGCCTTCATTAGCTC |
| | | GCTCCCCGCGCCGAGGCTGGGGGGGG |
| SEQ ID NO: 2 | cg19598567 | CCTCCCGCAGCTCATTGCAGCCCCGAGGAAATCACCGGGGGAGGGCTCG |
| | | GGAGTGCGGCG[CG]GCAGCCCCATAATTTCCAGGGCCCTTCTCCTACACT |
| | | GACACGTAATTGTCAGATTGTTTT |
| SEQ ID NO: 3 | cg13944175 | CCGCCGCGGGTTCCCAGGGCTGGTGGTAGTTGCCGTCCCACACGTACGT |
| | | GGCGGGGTCCT[CG]TCAGCGAAGACCTCGCGGAACATGTCGACCATGTA |
| | | GAGGTCCTCGGCGCGGTTGCCATCC |
| SEQ ID NO: 4 | cg19717586 | GGGGAGGAATATTAGACTCGGAGGAGTCTGCGCGCTTTTCTCCTCCCCG |
| | | CGCCTCCCGGT[CG]CCGCGGGTTCACCGCTCAGTCCCCGCGCTCGCTCCG |
| | | CACCCCACCCACTTCCTGTGCTCG |
| SEQ ID NO: 5 | cg22721334 | CAGGCCGGTCCCAGCCGCCCGGAGCCCCAGTGCGCGATGGCGGCCGGC |
| | | AAACTGCGCCTG[CG]CACTGGGCCTCACCGCGGACTACGACTCCCACAAT |
| | | GCCGCGAGGCTGTGCCGCGCACCGG |
| SEQ ID NO: 6 | cg13985485 | GTGACGCGCGGCCGCAGCTGCCCGCGGGCGGAGCGCTCTCAGACCCCG |
| | | GAGCGCACACCG[CG]GGGCCATCGGTGCCATCGCGGATCTCCAGGCTCC |
| | | TCATCAGTCCGCCGGGGCCGCAGCAG |
| SEQ ID NO: 7 | cg11358689 | GAGGAATATTAGACTCGGAGGAGTCTGCGCGCTTTTCTCCTCCCCGCGCC |
| | | TCCCGGTCGC[CG]CGGGTTCACCGCTCAGTCCCCGCGCTCGCTCCGCACC |
| | | CCACCCACTTCCTGTGCTCGCCC |
| SEQ ID NO: 8 | cg01944624 | ATCTACCGTCTCCAATCTCCATCTCCGAAGTTATGCCCACTTCCTCGAAGT |
| | | TTGGAGCCA[CG]CGAACTACACTGCCCAGAAGGCGCCGCGCCGTGAGCC |
| | | GCGATGCTTGGCCAATGAAAAGA |
| SEQ ID NO: 9 | cg04864807 | GGGAGGGCTCGTGAGAGCCAATGAGAGCGCGGAAGGCGGCGAGCGAG |
| | | CCAATGGACGCGG[CG]GTGGGGCAGGGGGCGGGGCCTGGGCGAGGCC |
| | | GGGGGCGGAATGGGCTGAGTGCCCTGTCT |
| SEQ ID NO: 10 | cg13849378 | CGGCAAGCGGAGCAGCGAGGCAGGGTAGCTTCATCACACTCGCGGCGG |
| | | ATGCGGATTCCG[CG]CCGCCCCGGCTCTAGCTGCTCAGGCGACCGCCAC |
| | | CCTCGCCTCGCCGCCGCCCGTGCACA |
| SEQ ID NO: 11 | cg19274890 | GCGGACGGCGGCTCCATCCGCGGCAATCACCGTAGTGCTTGTTTGTGGA |
| | | AGCCGAGCGTG[CG]TGCGCCGCGCGCGCACCCAGTCCAGCGCGGAGTG |
| | | GGCGTCTACCCGAGGAGGGGTGTCTG |
| SEQ ID NO: 12 | cg06783737 | TGGGGAATTAGCTCAGGCGGTGGAGCGCTCGCTTAGCTATGCGAGAGGT |
| | | AGCGAGATCGA[CG]CCCGCATTCTCCAGTTTCTTGTCTGGTTTATGTCTCT |
| | | TAGTTTGTATTCCCCGTTGTTTC |
| SEQ ID NO: 13 | cg19429281 | GAAGTCCCAGGGACCTGCGGAGCGCAGACATAACACAACACAGAGCAA |
| | | AACTCACCGCTG[CG]GTGACTTTCACTCCACGCGATCCGCTTCCCGGTTT |
| | | ACGCTAAACTGGGCGCTCGGGACAG |
| SEQ ID NO: 14 | cg00064733 | GGCTGCGGACGGCGGCTCCATCCGCGGCAATCACCGTAGTGCTTGTTTG |
| | | TGGAAGCCGAG[CG]TGCGTGCGCCGCGCGCGCACCCAGTCCAGCGCGG |
| | | AGTGGGCGTCTACCCGAGGAGGGGTG |
| SEQ ID NO: 15 | cg25258740 | CCCCCGCCGGCCGCCGGCCGCGCTCCCCGCCTTCATTCTGTGATCTGCGG |
| | | ATTTGCCAGT[CG]CCAACCTCCGCGCCCAGAGTCACCATCGCGCAGGGTT |
| | | GGGCAAACCATGGAGCTCGGGGC |
| SEQ ID NO: 16 | cg08087594 | AACTCCTGCACAAATCATTTCAAACGCGGTCGGCTTCTAATCGGGAAGTA |
| | | ATCTCAGTGA[CG]CTGGCGGTGCAGAGAACCGAGTCTGGACGCACACAC |
| | | ACAAACACACCGCGGGCCTCCGCA |
| SEQ ID NO: 17 | cg17233763 | GTGTGCTCAGCCTCAGCGTGAGGGGCACCTGCTCGTCTGGGCTCACAGC |
| | | GAAGGCAGCCT[CG]CCGCGAGCTGCCGCTGCCGCTGCTGCCGCCACTGG |
| | | TGTTGCCGCTCTCAGGCGCCAGGCT |
| SEQ ID NO: 18 | cg11372636 | GCCGGGAGCCTGACGTCACCACGCCCTGCCTGTCAATCTGCAGCGCGCG |
| | | CCGCTCGCAGC[CG]CCTTTTCTGCCACCAACTGTATCTCTCACTCGCGGA |
| | | GCCGGCACAGCGACAGGCGCCCCG |
| SEQ ID NO: 19 | cg01650149 | GCGGCGGCGGGGGGGAGCCAGGCCCGAGCTGCGTTCTGCGCAGCCAT |
| | | TGGTGGGCGCCG[CG]CTCTGCACTGAGCATGTTCGCGCCCCGCCGGCCC |
| | | CTAGCCGCAGCCGCAGCCGCAGCGAC |
| SEQ ID NO: 20 | cg17445666 | CAACCGGTTCCGCCGCGTTTGTGGGCTGGTAGCCCGGAATACATTTCCCA |
| | | GAGGCCTTCG[CG]GCCGACGTGCTTCGCGCAGGAACGCAGCCGCCTCCC |
| | | GACTGGAGGACGCGGTAGCGGAGC |
| SEQ ID NO: 21 | cg24415208 | GCTGCCCGTGGTCAAACTGGAGTCGCTGAAGCGCTGGAACGAAGAGCG |
| | | GGGCCTCTGGTG[CG]AGAAGGGGGTGCAGGTGCTGCTGACGACGGTGG |
| | | GCGCCTTCGCCGCCTTCGGCCTCATGA |
| SEQ ID NO: 22 | cg24221648 | CTTCCCGGCTCCCCGCGGTGCGCACCCGCTGGCCACTCTGCGCACGCGCG |
| | | CCGGGTGCCC[CG]GCCTAAGGCCGTTGACCTCGGGTTCTCCCCGGCACA |
| | | GTCGAATCCACGCCAGGGCCCTCA |
| SEQ ID NO: 23 | cg09017434 | GCGGGGGAGGTTGCGGGGGAGGCTCGGCGTCCCCGCTCTCCGCCCCGC |
| | | GACACCGACTGC[CG]CCGTGGCCGCCCTCAAAGCTCATGGTTGTGCCGC |
| | | CGCCGCCCTCCTGCCGGCCCGGCTGG |
| SEQ ID NO: 24 | cg15814717 | TGTACTACTTCCTCTGCCACCTGGCCTTGGTAGACGCGGGCTTCACTACTA |
| | | GCGTGGTGC[CG]CCGCTGCTGGCCAACCTGCGCGGACCAGCGCTCTGGC |
| | | TGCCGCGCAGCCACTGCACGGCC |
| SEQ ID NO: 25 | cg23619365 | AAAAAAAAAAAAAAGCAATGAGCCGCAAGCCTTGGACTCGCAGAGCTGC |
| | | CGGTGCCCGTC[CG]AGAGCCCCACCAGCGCGGCTCACGCCTCAGTCTCG |
| | | CCGCCCCAAGGTGGGATCCGACGCC |
| SEQ ID NO: 26 | cg20457275 | CGAGAGGGCCCGGTCCAGCAGCCTCTGGGGCCCAGTGCGCAGGGCACT |
| | | GCGGGCCGATTG[CG]CCCCGGGGCCAGGAGGCGCCGAGAAAGCAAAA |
| | | GCAAAAGCCGGCGGCGGGTGGAGGTCAA |
| SEQ ID NO: 27 | cg22305167 | CGGCCGCAGTGTGCCGCCCGCTGCGCTATGCGGGGCTCGTCTCCCCGCG |
| | | CCTATGTCGCA[CG]CTGGCCAGCGCCTCCTGGCTAAGCGGCCTCACCAAC |
| | | TCGGTTGCGCAAACCGCGCTCCTG |
| SEQ ID NO: 28 | cg16664405 | CCTGGCGCGACCGCCAGCAGCACCCAGCGCGGGGCCGGGAGCTGCTGG |
| | | GGGCCCAGGCTC[CG]CTCTCCCCACCGCTCTGCACCGCTGCCGGCTGCG |
| | | GACAGACCCGATGCGCCACCACCACC |
| SEQ ID NO: 29 | cg16585333 | CCGGAGCGCGCTGCTGCCCTCTACCGGTCATCCGTGCGGCCGGACACCG |
| | | TGTCAGGCCCG[CG]AGGAGGGCTCTGCCGCAGTCCCGGGGAACAGCAC |
| | | CCAGCAGCGCCACTGGGAGAGGAAAC |
| SEQ ID NO: 30 | cg05057720 | AGTCCAGAGCGGCGCTGTGCAGCTGGAAGGGCGCGCGATAGCTCAAGT |
| | | TAGAGGCGGCCC[CG]GGGCGCGGCGCAGGACACAAGACCTCAAACTGG |
| | | TACTTGCACAGGTAGCCGTTGGCGCGC |
| SEQ ID NO: 31 | cg03419058 | GGCGGTGCGAGCTCCCCGCCTGCGGGACGCACGGAGACCGCGGTCAGC |
| | | GCGCCGCCTGGC[CG]GCCCAGCGCGCCCAGCCCGCGCCCAGCCCCGTCC |
| | | ACTCCCGTCCAGCCCCGCCGCCCGGC |
| SEQ ID NO: 32 | cg02473540 | CGGTAGAGTTTCCAACACGAAAGCCCGTGTGGTCGCGCCGGGAGCTCAC |
| | | GGCGTTCCAAG[CG]GCACTTATCCCGCGTTGATGCCCAGGCACCCCGCG |
| | | CGCCCTGTTTCACCAGGCCCAGTCA |
| SEQ ID NO: 33 | cg01758512 | CCAGCGGCAGTAGCTGTAGCAGCTTCAGCGAAGCCGGAGATGGGCAGA |
| | | GAGCGCGCGCGG[CG]CAGCAGCTCCAGATTCACTGCTCTCCCCTGCAGC |
| | | TCCCCGCGCCCCCGCCGCTGTCGCTG |
| SEQ ID NO: 34 | cg18897632 | GTGTTCTCTGCGGCGGGCCGCGTCCCCGCTGAGCCTCGCGGTGACAGCC |
| | | GCCTTTGGCAG[CG]AGCGCTCGGGGCACTTCTATCCCCGCCTCTCAAAGG |
| | | GTGGGGACAGCCGTTTCCAGATTT |
| SEQ ID NO: 35 | cg09568464 | CGGCCGCGCCCCCGGCAGCCCAGGGCGCGCTTCCACCACGGTACCGGTG |
| | | GATTCGCCGTG[CG]CAGCCGGAAGATGGCGCAGACGCACAAAGCACAC |
| | | CGATGCTGCGCCATGATAGGGCCGGC |
| SEQ ID NO: 36 | cg15811515 | TCTCGCGGCGCAGGCGGCGGCGGCAGAGGTGGGGTCGCGCAGCGGAG |
| | | GCAGCTCGAGCTT[CG]GGATGCGCGCTCGCTTCTTGGGCTCCTCGCTCGA |
| | | TCTTACTGCCCCCTTTTTTCTCTCCC |
| SEQ ID NO: 37 | cg00884040 | TCCTCCAGCCAGAGTCGGTGGGACTGGCTGCGCTGCCCTGAAGTGGTTC |
| | | TCCAAGCAGCG[CG]GAGGGTGGCGGACGGCGGACGGAGCCCAGGGGC |
| | | CGCGTCGGGTGGGGAAACCCGAACTCG |
| SEQ ID NO: 38 | cg21632158 | TGCGCATCGCTGGCTCTGGGTTCCGCCGAATGCGTCCTCCTGGCGGTGAT |
| | | GGCTCTGGAC[CG]CGCGGCCGCAGTGTGCCGCCCGCTGCGCTATGCGGG |
| | | GCTCGTCTCCCCGCGCCTATGTCG |
| SEQ ID NO: 39 | cg18343957 | AGGGGAGCTGCGAGGCGAAGTGTTCTTCAGGGAAGCGGGCTCGAGTCT |
| | | CCGCAGCTGCGG[CG]GCGGCGGCGGCGCGCTGGGCCGGCGGCGGGCG |
| | | CGGGCAGGGGGCCGGGGGTGCCGCGCGG |
| SEQ ID NO: 40 | cg23883696 | CCTCCACCCCCGGGGGGTTCCTGCGCACTGAAAGACCGTTCTCCGGCAG |
| | | GTTTTGGGATC[CG]GCGACGGCTGACCGCGCGCCGCCCCCACGCCCGGT |
| | | TCCACGATGCTGCAATACAGAAAGT |
| SEQ ID NO: 41 | cg24403845 | AGAGAGGGGTCCCAGAACGAAGGTGGCGGCACGAGCTCTGCGCTGGCG |
| | | GCTGTGGGGGGC[CG]GCGCTCAGGACCCCAACTCCATCCAAGTTGCGCC |
| | | GCGGTGGGGGGGGGGGGAGGCGGCGC |
| SEQ ID NO: 42 | cg20405017 | AATCTCCCCTCGGGCTCGACGGATGTGCGCCCCAGATGTGCTGACACATG |
| | | TCCGATGCCT[CG]CTGCCTTGGAGGTCTCCCCGCTCGCGTGTCTCTTCTCT |
| | | TCGCACCAGCGGCGGAAACCGC |
| SEQ ID NO: 43 | cg21678377 | GCTCCGCTTCTCCGGGTTTTAGCGGAAGCCTGCGGGGGGCGGGGTAACC |
| | | GCGGAAGCCGG[CG]GCCGTGGGCGCGCGGGTTGGGGGCTCTCGCGCCG |
| | | CTCCGGGCTCTCCCCCCCCCCGGCTG |
| SEQ ID NO: 44 | cg03753331 | CGCGCTCCGCTTCTCCGGGTTTTAGCGGAAGCCTGCGGGGGGCGGGGTA |
| | | ACCGCGGAAGC[CG]GCGGCCGTGGGCGCGCGGGTTGGGGGCTCTCGCG |
| | | CCGCTCCGGGCTCTCCCCCCCCCCGG |
| SEQ ID NO: 45 | cg16587616 | GCGAGGGATCTCTGTGCGTCCTCACTGGCCCATGCACCCAGCACCTGCGA |
| | | CTCCCGCCGT[CG]GGCTGCGTGGCCCCGCGCCCACACCTGCCCGTCCCTT |
| | | CCGTCGTCCCTCGCTCGCGCAGA |
| SEQ ID NO: 46 | cg25730685 | GGGGAGGTGTGGGGAGCGGAAGGCCGCAGGAGCATCTTTGCGGAGAA |
| | | AGTACTTTGGCTG[CG]GCGGGCGCAGGGCGGGCCGGCTAGCCCCGCGC |
| | | CCCACCTGTTCTGTGCGTCGCGCTCGCC |
| SEQ ID NO: 47 | cg20019985 | TAGGGCTGGAAACCCGCCGCCACAGCGGGCTAGAGGTCGTCCCCGCCCG |
| | | CAACATATGCG[CG]AAGGAAAGTGCTACGAACGTCAAATGGCCGCCCCC |
| | | CGCCGACGCCATCTGCTCTGCGAAG |
| SEQ ID NO: 48 | cg03730428 | CGCCCGCAACATATGCGCGAAGGAAAGTGCTACGAACGTCAAATGGCCG |
| | | CCCCCCGCCGA[CG]CCATCTGCTCTGCGAAGCAGAAACGGCGGCAGCTG |
| | | CGCGCCCAGTCCCTCCGCCCGCGCC |
| SEQ ID NO: 49 | cg18384778 | CCCCCTGTTCAAGGTCTGTCACCGTAGGGGGGGGGGGGGCGCGTGGAG |
| | | CCGCTGGGGGTT[CG]GCCCACCCCGCGAACCGAGCTCCCGGCCCTGTGC |
| | | GCCCTCAGCTCTGCCGCGGGCGTTGG |
| SEQ ID NO: 50 | cg22010052 | GCTGTGGCCGCAGCTGAGGCCCGACGAGCTTCCGGCCGGGTCTTTGCCC |
| | | TTCACTGGCCG[CG]TGAACATCACGGTGCGCTGCACGGTGGCCACCTCTC |
| | | GACTGCTGCTGCATAGCCTCTTCC |
| SEQ ID NO: 51 | cg19688250 | GTGTGCGTGTGCGTGTGCTCAGCCTCAGCGTGAGGGGCACCTGCTCGTC |
| | | TGGGCTCACAG[CG]AAGGCAGCCTCGCCGCGAGCTGCCGCTGCCGCTGC |
| | | TGCCGCCACTGGTGTTGCCGCTCTC |
| SEQ ID NO: 52 | cg04701034 | TGGGGCAGCGGCGTTGCAGGAGATGAGCTCAGCGCAAAGGGAACCCCG |
| | | CAGCGGCGAGTG[CG]GCTGCTGGCCTGCGCGCTGTGGCCCCAACAGGCT |
| | | GGCAGGGCGCGGGCGGGTGGCGGGGT |
| SEQ ID NO: 53 | cg20505704 | AGAGTCGGTGGGACTGGCTGCGCTGCCCTGAAGTGGTTCTCCAAGCAGC |
| | | GCGGAGGGTGG[CG]GACGGCGGACGGAGCCCAGGGGCCGCGTCGGGT |
| | | GGGGAAACCCGAACTCGCGGAGGGGAA |
| SEQ ID NO: 54 | cg15124215 | AAAGCCCTGGCAGGTAAAGAGAGGACCCGCGCAGGCTGGGAGCTCCCA |
| | | CTCCTCCTCCAG[CG]TCACGCTCGCCCTCCGCCGCTGCCTCGCGTCCGGG |
| | | TCTGTTTATATAGCGTCTGGAGGCC |
| SEQ ID NO: 55 | cg07143083 | CTGGCCAAGTGCCGGCCCATCGCGGTGCGCAGCGGAGACGCCTTCCACG |
| | | AGATCCGGCCG[CG]CGCCGAGGTGGCCAACCTCAGCGCGCACAGCGCC |
| | | AGCCCCATCCAGGATGCGGTCCTGAA |
| SEQ ID NO: 56 | cg00688962 | GGCGCCGGCAGCTTCGCGCCGGCGGCTGGAAGCGGGCGGGCTGCACGG |
| | | GCGGCTCGAGTG[CG]GGGACCCCAGCCCCTCGCCCTCGTGAGCGCCGCC |
| | | CCTGCCACCTGCTGCCAAGTCACCGG |
| SEQ ID NO: 57 | cg00027083 | CCCCGGCCGCGCCGGGCGCGGGGCTCGGGATTCGGGAGACCGCGCGGC |
| | | GCCGAAGCCACG[CG]TCAGCCCCACTGTCCCGCGCGCCTCGCCCCAGGC |
| | | CTCGGGCTCTTCCTCCGCACCTCGTA |
| SEQ ID NO: 58 | cg08305436 | ACGCGGGGACTGGAAAGGGCGCCTGGGTGGGAAGAGGCGCTGGCGGG |
| | | TGATCGTCCCCAC[CG]GGCCAGTCCCCGGGATCTGCTGCCGCCCCTCTCC |
| | | GAAATTCACAGCCAGAGCGGGCGCAC |
| SEQ ID NO: 59 | cg14638883 | TCTGAGAAGTGTCCTCCTCGCTCTCTTATAAAAACAGGACTTGTTGCCGA |
| | | GGTCAGCGCG[CG]CATCGAGTGTGCCAGGCGTGTGCGTGGTTTCTGCTG |
| | | TGTCATTGCTTTCACGGAAGGTGG |
| SEQ ID NO: 60 | cg09907509 | GCGCCCAGACTGCGCGCCGCGCCGCTGCGCCCAACATTCCCGAGGACGG |
| | | CTTCGCGGGCG[CG]TATCGTCCAGACCGGAGCACCGCCCCACCGCTAGC |
| | | GCAGGAGACCTGCCGGGGAAGTCGC |
| SEQ ID NO: 61 | cg20707222 | AAAGGCCGTACTCTGCCCCCCGCGGGACCCAGGTCCCCGCCTGCTGCAG |
| | | AGCGCACTCTG[CG]CACGTCGAGCCGCGAAAGGTTCACAGAAGAAAACA |
| | | AGAGAAAGAAGTAGCAGGCACTGAG |
| SEQ ID NO: 62 | cg17056618 | GGAATCCATTCTTTTAAGCCAGGGTTTAAAACTCTTCAAGCAAGTCATCT |
| | | GCAAAGGTAC[CG]CTTCTACCATTITAAAGATAGGATTATGTTCCCTAGG |
| | | ACAACTGGATGAGCCCTAGGAAC |
| SEQ ID NO: 63 | cg18058689 | GAGGAGCGCGCCGCTGCCTCTGGCGGGCTTTCGGCTTGAGGGGCAAGG |
| | | TGAAGAGCGCAC[CG]GCCGTGGGGTTTACCGAGCTGGATTTGTATGTTG |
| | | CACCATGCCTTCTTGGATCGGGGCTG |
| SEQ ID NO: 64 | cg22620221 | CCCTGTGCGTGCCGCCGCGCTGTTGCTCGCAGTGTGCTGGCGCCGAGCTC |
| | | GGTGGACACG[CG]CGCAGTCAGAGCTGCCTCTCGCCCTCGCTAGCTGGG |
| | | CTCGCAGCCTCTTCCTCCCTCCCT |
| SEQ ID NO: 65 | cg02547394 | CTCTTTGGCAAGTGGTTTGTGCATCAGGAGAAACTTTCCACCTGCGAGCC |
| | | GAACCGGCGC[CG]AGTGCGTGTGTTTCTGCCTTTTTTTGTTGTCGTTGCCT |
| | | CCACCCCTCCCCATTCTTCTCT |
| SEQ ID NO: 66 | cg09469566 | TGGCTGCCAGAGCGAGTGAGGGGCGCAGAGGCGGCAGAGAGCGGAGA |
| | | GCCCCGGTGTCTC[CG]CGAGGGCGGCGGCGGCCAGCAGACGGCGATCG |
| | | AGGCGCGCGCCACGGCACGGCCAGCGCA |
| SEQ ID NO: 67 | cg26609631 | AAGCGCGTGGAGAGCCGAAAGGTGCGGTGGGCGCAGAGGGCGGGCTG |
| | | GCTGCGGGGCGAC[CG]CGCGCCGGGGCCATGCCGCGCTCCTTCCTGGTG |
| | | GACTCGCTAGTGCTGCGCGAGGCGGGC |
| SEQ ID NO: 68 | cg10132208 | GGGGTCGCCATGACCGAGTGGCCCAGGCCCGAGCGAAGCCCGCGCGCG |
| | | GTGAGTCCGCCG[CG]GCCCATCCGTCCCTCCGCCCGCCAGAGCGTCCATC |
| | | GGGACGCCCACCCGGGAGGGTCTCG |
| SEQ ID NO: 69 | cg06000994 | CCGAGCGCTGCCCCCGCCGGCCCGCGGCTGCCAGCCGGCCCTGCCCGCG |
| | | CCCGGGCCCCG[CG]AGCGGCCGCACTTCACCTTACGGAGGGGAGATAAT |
| | | GAGATCAATTAGAGGCGCCGTCACC |
| SEQ ID NO: 70 | cg10182317 | GGCAACCCTGACTCGGACCGCTCGGGAGAGCCCCAGGAGAGGCCAGCG |
| | | CCGCGCAGCAGC[CG]CCCCGCTGCGCCCACCTCCCCGGCTGCTCCCGGA |
| | | GGGCTCACAAAGGCGGTGGCCGCCCG |
| SEQ ID NO: 71 | cg14222229 | GCGGGCGGCAGCCGCAAGCGAGGAATCCAGCGCAGGGAAAGTAGCCCC |
| | | AGTGGGGCCCGG[CG]CGTCAGCCCCACTCGCGTGGCAAAACTTGCGGG |
| | | GGCCCCCGCGTGCCGCGCCTCAGCCCA |
| SEQ ID NO: 72 | cg04596005 | TCCTCGCCGTCGGGGTCCTCCTCCTCTGCCGACGAGTTGTCACTGGGCGA |
| | | GGCGTAGCTG[CG]CTCTACGCCGCGGAGGGGCGGCCTCTTGGAGGCGG |
| | | GGACCGGGTACTCCCGCTGCAGCCC |
| SEQ ID NO: 73 | cg11592503 | GCTGCTCGCGCTCCGCCGCCCGGGAGATGCTTCCTCGCGCGGCGCAGCG |
| | | CTGAGGCCGTG[CG]TGCGCCCCGGCTGCGCTGCGCGCTCCCCACATACA |
| | | CAAGCTCTCCATGTGAGCTGACAGG |
| SEQ ID NO: 74 | cg05008595 | CTTCTCTTGAAAAGGAGGAGAATCAACACTGGGCTCACAACTCATCAGA |
| | | GCTGAGTCATA[CG]TACATCAGCAGGACCTACGTGGGAACCAAATAGCA |
| | | AACTCAAATTGGGAAATTTGAGGAA |
| SEQ ID NO: 75 | cg04999026 | CCGAGAGCCCCGCCTGCAGGCGGTGTAGATACATGTAGATACTGTAGAT |
| | | ACTGTAGATAC[CG]CCCCGGCGCCGACTTGATAAACGGTTTCGCCTCTTT |
| | | TGGAAGCCGCCTGCGTGTCCATTT |
| SEQ ID NO: 76 | cg04546413 | TGAGGAGTGAGGAGGCAGAAAGGACCGAGAACAAGGGGACCCGGTTC |
| | | CATTTCTGGACCC[CG]TCCGCAGGCTGCTCGCCCGACTTGGGGTCGCTCT |
| | | GCCCCGGACGATCAGGACAGCTGCGT |
| SEQ ID NO: 77 | cg27254667 | CAAATCTATATGAAGGATCGAATTGCATTGAACTAGCAAACACACACACA |
| | | CACACGCACA[CG]CAAAAACTGATGAAAGCTGAACAAGGTCTGTAGTCT |
| | | AGTCAACAGTACTGCACTATGTGA |
| SEQ ID NO: 78 | cg18902440 | ACAGTCTCTCGCCTCAAAGATCTCCGCCATTAGTGGTAGCCATTTAAGAA |
| | | AACAGAATTA[CG]ATGAATAATGATTTGAAGCCAAAAAGTCAAAATATCT |
| | | TATTTCGCAACTGTAATTGCTGG |
| SEQ ID NO: 79 | cg01315092 | CCACACAGGCCTCTCCCTCGGTGCGGTAGCGAGGGTTGCGGGCCCAAAC |
| | | GCCCGCGCCCA[CG]GAGGCGCCTGCGACGACTAGAAGCTTCCACAGCCA |
| | | TATGGGGGCAAAGACGGCCCAGTAG |

In an embodiment of the present invention, it discloses a panel of DNA methylation biomarkers for screening and early detection of cervical cancer, wherein the panel comprises of CGIDs derived by the APDMA method having sequences selected from the group consisting of SEQ ID NO:1 to SEQ ID NO:79, and optionally said panel is used in combination with other biomarkers as early predictors of cervical cancer.

In an embodiment of the present invention, the polygenic DNA methylation biomarkers are a combination of CGIDs in the list below in Table 2 or a short subset of this list such as the example listed below in Table 3 for early detection of cervical cancer and risk of cervical cancer in women with CIN1 to CIN3 precancerous lesions.

Thus, in an additional embodiment of the present invention, it discloses an in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said candidate CGIDs as the early predictors of cervical cancer as DNA methylation biomarkers, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70.

### Table 2: Selected Subset of Polynucleotides from Table 1 having CpG Methylation Sites Useful in Embodiments of the Present Invention

The 16 CGID biomarkers discussed herein are found in Table 2 that is included with this application. These 16 shortlisted DNA methylation biomarkers were hypermethylated between CIN3 and CIN1 and control, with highest effect size (Cohen D>1.3) between CIN3 and control and highest Spearman correlation with progression of CIN phases r>0.4.

| SEQ ID NO | Probe | Probe with CpG marked |
|---|---|---|
| SEQ ID NO: 3 | cg13944175 | CCGCCGCGGGTTCCCAGGGCTGGTGGTAGTTGCCGTCCCACACGTACGT |
| | | GGCGGGGTCCT[CG]TCAGCGAAGACCTCGCGGAACATGTCGACCATGTA |
| | | GAGGTCCTCGGCGCGGTTGCCATCC |
| SEQ ID NO: 4 | cg19717586 | GGGGAGGAATATTAGACTCGGAGGAGTCTGCGCGCTTTTCTCCTCCCCGC |
| | | GCCTCCCGGT[CG]CCGCGGGTTCACCGCTCAGTCCCCGCGCTCGCTCCGC |
| | | ACCCCACCCACTTCCTGTGCTCG |
| SEQ ID NO: 7 | cg11358689 | GAGGAATATTAGACTCGGAGGAGTCTGCGCGCTTTTCTCCTCCCCGCGCC |
| | | TCCCGGTCGC[CG]CGGGTTCACCGCTCAGTCCCCGCGCTCGCTCCGCACC |
| | | CCACCCACTTCCTGTGCTCGCCC |
| SEQ ID NO: 17 | cg17233763 | GTGTGCTCAGCCTCAGCGTGAGGGGCACCTGCTCGTCTGGGCTCACAGC |
| | | GAAGGCAGCCT[CG]CCGCGAGCTGCCGCTGCCGCTGCTGCCGCCACTGG |
| | | TGTTGCCGCTCTCAGGCGCCAGGCT |
| SEQ ID NO: 19 | cg01650149 | GCGGCGGCGGGCGGGGAGCCAGGCCCGAGCTGCGTTCTGCGCAGCCAT |
| | | TGGTGGGCGCCG[CG]CTCTGCACTGAGCATGTTCGCGCCCCGCCGGCCC |
| | | CTAGCCGCAGCCGCAGCCGCAGCGAC |
| SEQ ID NO: 31 | cg03419058 | GGCGGTGCGAGCTCCCCGCCTGCGGGACGCACGGAGACCGCGGTCAGC |
| | | GCGCCGCCTGGC[CG]GCCCAGCGCGCCCAGCCCGCGCCCAGCCCCGTCC |
| | | ACTCCCGTCCAGCCCCGCCGCCCGGC |
| SEQ ID NO: 34 | cg18897632 | GTGTTCTCTGCGGCGGGCCGCGTCCCCGCTGAGCCTCGCGGTGACAGCC |
| | | GCCTTTGGCAG[CG]AGCGCTCGGGGCACTTCTATCCCCGCCTCTCAAAGG |
| | | GTGGGGACAGCCGTTTCCAGATTT |
| SEQ ID NO: 39 | cg18343957 | AGGGGAGCTGCGAGGCGAAGTGTTCTTCAGGGAAGCGGGCTCGAGTCT |
| | | CCGCAGCTGCGG[CG]GCGGCGGCGGCGCGCTGGGCCGGCGGCGGGCG |
| | | CGGGCAGGGGGCCGGGGGTGCCGCGCGG |
| SEQ ID NO: 42 | cg20405017 | AATCTCCCCTCGGGCTCGACGGATGTGCGCCCCAGATGTGCTGACACATG |
| | | TCCGATGCCT[CG]CTGCCTTGGAGGTCTCCCCGCTCGCGTGTCTCTTCTCT |
| | | TCGCACCAGCGGCGGAAACCGC |
| SEQ ID NO: 43 | cg21678377 | GCTCCGCTTCTCCGGGTTTTAGCGGAAGCCTGCGGGGGGCGGGGTAACC |
| | | GCGGAAGCCGG[CG]GCCGTGGGCGCGCGGGTTGGGGGCTCTCGCGCCG |
| | | CTCCGGGCTCTCCCCCCCCCCGGCTG |
| SEQ ID NO: 49 | cg18384778 | CCCCCTGTTCAAGGTCTGTCACCGTAGGGGGGGGGGGGGCGCGTGGAGC |
| | | CGCTGGGGGTT[CG]GCCCACCCCGCGAACCGAGCTCCCGGCCCTGTGCG |
| | | CCCTCAGCTCTGCCGCGGGCGTTGG |
| SEQ ID NO: 56 | cg00688962 | GGCGCCGGCAGCTTCGCGCCGGCGGCTGGAAGCGGGCGGGCTGCACGG |
| | | GCGGCTCGAGTG[CG]GGGACCCCAGCCCCTCGCCCTCGTGAGCGCCGCC |
| | | CCTGCCACCTGCTGCCAAGTCACCGG |
| SEQ ID NO: 57 | cg00027083 | CCCCGGCCGCGCCGGGCGCGGGGCTCGGGATTCGGGAGACCGCGCGGC |
| | | GCCGAAGCCACG[CG]TCAGCCCCACTGTCCCGCGCGCCTCGCCCCAGGCC |
| | | TCGGGCTCTTCCTCCGCACCTCGTA |
| SEQ ID NO: 58 | cg08305436 | ACGCGGGGACTGGAAAGGGCGCCTGGGTGGGAAGAGGCGCTGGCGGG |
| | | TGATCGTCCCCAC[CG]GGCCAGTCCCCGGGATCTGCTGCCGCCCCTCTCC |
| | | GAAATTCACAGCCAGAGCGGGCGCAC |
| SEQ ID NO: 65 | cg02547394 | CTCTTTGGCAAGTGGTTTGTGCATCAGGAGAAACTTTCCACCTGCGAGCC |
| | | GAACCGGCGC[CG]AGTGCGTGTGTTTCTGCCTTTTTTTGTTGTCGTTGCCT |
| | | CCACCCCTCCCCATTCTTCTCT |
| SEQ ID NO: 70 | cg10182317 | GGCAACCCTGACTCGGACCGCTCGGGAGAGCCCCAGGAGAGGCCAGCG |
| | | CCGCGCAGCAGC[CG]CCCCGCTGCGCCCACCTCCCCGGCTGCTCCCGGAG |
| | | GGCTCACAAAGGCGGTGGCCGCCCG |

In an embodiment of the present invention, it discloses a combination of DNA methylation biomarkers for screening and early detection of cervical cancer, said combination comprises of CGIDs derived using the APDMA method, for detecting cervical cancer by measuring DNA methylation levels of said CGIDs in DNA originating from cervical specimens and deriving a “cervical cancer methylation predictor” using linear regression equations and Receiver operating characteristics (ROC) assays, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70.

### Table 3: Selected Subset of Polynucleotides from Table 2 Having CpG Methylation Sites Useful in Embodiments of the Invention.

The 2 CGID biomarkers discussed herein are found in Table 3 that is included with this application. The subset in Table 3 represents the minimal number of CGID biomarkers that differentiate CIN3 premalignant lesions from control identified using a penalized regression which reduced the number of CGIDs to 5, followed by a multivariable linear regression with these 5 CGIDs as independent variables and CIN3 state as dependent variable. A linear regression equation composed of weighted methylation levels of these two sites was highly significant for prediction of CIN3 (p<5×10⁻¹⁵).

| SEQ ID NO | Probe | Probe with CpG marked |
|---|---|---|
| SEQ ID NO: | cg13944175 | CCGCCGCGGGTTCCCAGGGCTGGTGGTAGTTGCCGTCCCACACGTACGT |
| 3 | | GGCGGGGTCCT[CG]TCAGCGAAGACCTCGCGGAACATGTCGACCATGTA |
| | | GAGGTCCTCGGCGCGGTTGCCATCC |
| SEQ ID NO: | cg03419058 | GGCGGTGCGAGCTCCCCGCCTGCGGGACGCACGGAGACCGCGGTCAGC |
| 31 | | GCGCCGCCTGGC[CG]GCCCAGCGCGCCCAGCCCGCGCCCAGCCCCGTCC |
| | | ACTCCCGTCCAGCCCCGCCGCCCGGC |

In an embodiment of the present invention, it discloses a combination of DNA methylation biomarkers for screening and early detection of cervical cancer, said combination comprises of CGIDs derived using the APDMA method, for detecting cervical cancer by measuring DNA methylation levels of said CGIDs in DNA originating from cervical specimens and deriving a “cervical cancer methylation predictor” using linear regression equations and Receiver operating characteristics (ROC) assays, wherein the said CGIDs are as set forth in SEQ ID NO:3, and SEQ ID NO:31.

In an embodiment, the present invention provides a kit and a process for detecting cervical cancer, comprising means and reagents for detecting DNA methylation measurements of a panel of polygenic DNA methylation biomarkers for cervical cancer.

In an embodiment, the present invention provides a kit for detecting cervical cancer comprising means and reagents for DNA methylation measurements of the CGID biomarkers of Table 1.

In an embodiment, the present invention provides a kit for detecting cervical cancer, comprising means and reagents for DNA methylation measurements of CGIDs and deriving a DNA methylation predictor of cervical cancer and an instruction manual, wherein the CGIDs are as set forth in SEQ ID NO:1 to SEQ ID NO:79.

In an embodiment, the present invention provides a panel of CGIDs in the form of a chip for detecting cervical cancer, wherein the panel of CGIDs are as set forth in SEQ ID NO:1 to SEQ ID NO:79.

In an embodiment, the present invention provides a use of CGID biomarkers disclosed in the present invention.

In an embodiment, the present invention provides a use of DNA pyrosequencing methylation assays for predicting cervical cancer by measuring DNA methylation of CGIDs, wherein the CGIDs are as set forth in SEQ ID NO:1 to SEQ ID NO:79.

In an embodiment, the present invention provides use of DNA pyrosequencing methylation assays for predicting cervical cancer using CGID biomarkers listed above, for example using the below disclosed primers and standard conditions of pyrosequencing reactions recommended by the manufacturer (Pyromark, Qiagen):

cg03419058
SEQ ID NO: 80-Forward (biotinylated):
GGTTTTTGGGTAGGAAGGATAGTAG
SEQ ID NO: 81-Reverse:
AAACAAATCTAACCCCTAAAAAAAC
SEQ ID NO: 82-Sequence:
CAAACTAAACACACTAAACC
cg13944175
SEQ ID NO: 83-Forward:
GGGTTTTTAGGGTTGGTGGTA
SEQ ID NO: 84-Reverse (biotinylated):
TCCTCATAATAATAAATAACAACC
SEQ ID NO: 85-Sequence:
TATGTATGTGGTGGGGTT

In an embodiment, the present invention provides the use of DNA pyrosequencing methylation assays for predicting cervical cancer by measuring DNA methylation of CGIDs combinations, wherein the forward, biotinylated primer is as set forth in SEQ ID NO:80, the reverse primer is as set forth in SEQ ID NO:81, and the pyrosequencing primer is as set forth in SEQ ID NO:82.

In an embodiment, the present invention provides the use of DNA pyrosequencing methylation assays for predicting cervical cancer by measuring DNA methylation of CGIDs combinations, wherein the forward, biotinylated primer is as set forth in SEQ ID NO:83, the reverse primer is as set forth in SEQ ID NO:84, and the pyrosequencing primer is as set forth in SEQ ID NO:85.

In an embodiment, the present invention provides use of polygenic multiplexed amplicon bisulfite sequencing DNA methylation assay for predicting cervical cancer in cervical specimens' DNA by using CGID biomarkers listed above. For example, using the below disclosed primers and standard conditions that involve bisulfite conversion, sequential amplification with target specific primers (PCR 1) followed by barcoding primers (PCR 2) and multiplexed sequencing in a single next generation Miseq sequencer (Illumina), demultiplexing using Illumina software, data extraction and quantification of methylation using standard methods for methylation analysis such as Methylkit, followed by calculation of the weighted DNA methylation score and prediction of cancer.

The first PCR is performed as follows:

For CGID cg03419058
SEQ ID NO: 80-Forward primer:
5′ GGTTTTTGGGTAGGAAGGATAGTAG 3′
SEQ ID NO: 81 Reverse primer:
5′ AAACAAATCTAACCCCTAAAAAAAC 3′
For CGID cg13944175
SEQ ID NO: 83-Forward primer:
5′ GGGTTTTTAGGGTTGGTGGTA 3′
SEQ ID NO: 84-Reverse primer:
5′ TCCTCATAATAATAAATAACAACC 3′

To barcode (index) the samples, the present invention used a second PCR reaction with the following primers:

SEQ ID NO: 86-Forward primer:
5'AATgATACggCgACCACCgAgATCTACACTCTTTCCCTACACgAC3′

SEQ ID NO:87-Barcoding primer (reverse): 5′ CAAgCAgAAgACggCATACgAgATAGTCATCG gTgACTggAgTTCAgACgTg3′ (where the red bases are the index; and 1200 variations of this index are used)

In an embodiment, the present invention provides a use of multiplexed targeted-amplification bisulfite sequencing methylation assays on a next generation sequencer for detecting cervical cancer by measuring the DNA methylation levels of CGIDs combinations, wherein the CGIDs are as set forth in SEQ ID NO:1 to SEQ ID NO:79.

In another embodiment, the present invention provides a use of multiplexed targeted-amplification bisulfite sequencing methylation assays on a next generation sequencer for detecting cervical cancer by measuring the DNA methylation levels of CGIDs combinations, wherein the CGID as set forth in SEQ ID NO:3 has primers as set forth in SEQ ID NO:88 for the forward primer and SEQ ID NO:89 for the reverse primer.

In another embodiment, the present invention provides a use of multiplexed targeted-amplification bisulfite sequencing methylation assays on a next generation sequencer for detecting cervical cancer by measuring the DNA methylation levels of CGIDs combinations, wherein the CGID as set forth in SEQ ID NO:31 has primers as set forth in SEQ ID NO:90 for the forward primer and SEQ ID NO:91 for the reverse primer.

In an embodiment, the present invention provides use of Receiver operating characteristics (ROC) assays for detecting cancer by defining a threshold value between cervical cancer and normal cervix using weighted DNA methylation measurements of CGID biomarkers in Table 1 or a subset of these CGIDs such as in Table 2 as an example. Samples above the threshold are to be classified as cancer.

In an embodiment, the present invention provides the use of hierarchical Clustering analysis assays for predicting cancer with use in obtaining cancer positive early detection by using measurements of methylation of CGID biomarkers listed in Table 1.

In an embodiment, the present invention provides a use of mass spectrometry based (Epityper™) or PCR based methylation assays of DNA extracted from sample for detecting cancer by measuring the DNA methylation levels of CGIDs combinations as set forth in a panel of DNA methylation biomarkers for screening and early detection of cervical cancer, wherein the panel comprises of CGIDs derived by the APDMA method having sequences selected from the group consisting of SEQ ID NO:1 to SEQ ID NO:79, and optionally said panel is used in combination with other biomarkers as early predictors of cervical cancer.

In an embodiment, the present invention provides a use of multivariable linear regression equation or neural network analysis for calculating a methylation score predicting cervical cancer by using measurements of DNA methylation CGIDs combinations as set forth in a panel of DNA methylation biomarkers for screening and early detection of cervical cancer, wherein the panel comprises of CGIDs derived by the APDMA method having sequences selected from the group consisting of SEQ ID NO:1 to SEQ ID NO:79, and optionally said panel is used in combination with other biomarkers as early predictors of cervical cancer.

In an embodiment, the present invention provides a use of multivariable linear regression equation or neural network analysis for calculating a methylation score predicting cervical cancer by using measurements of DNA methylation CGIDs combinations as set forth in a combination of DNA methylation biomarkers for screening and early detection of cervical cancer, said combination comprises of CGIDs derived using the APDMA method, for detecting cervical cancer by measuring DNA methylation levels of said CGIDs in DNA originating from cervical specimens and deriving a “cervical cancer methylation predictor” using linear regression equations and Receiver operating characteristics (ROC) assays, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70.

In an alternate embodiment, the present invention provides a use of multivariable linear regression equation or neural network analysis for calculating a methylation score predicting cervical cancer by using measurements of DNA methylation CGIDs combinations as set forth in a combination of DNA methylation biomarkers according to claim 9, said CGIDs are as set forth in SEQ ID NO:3, and SEQ ID NO:31.

In an embodiment, the present invention provides a use of Receiver operating characteristics (ROC) assays to define a “methylation score” threshold differentiating cervical cancer from noncancer cervical tissue by using measurements of DNA methylation combinations as set forth in a panel of DNA methylation biomarkers for screening and early detection of cervical cancer, wherein the panel comprises of CGIDs derived by the APDMA method having sequences selected from the group consisting of SEQ ID NO:1 to SEQ ID NO:79, and optionally said panel is used in combination with other biomarkers as early predictors of cervical cancer.

In an embodiment, the present invention provides a use of Receiver operating characteristics (ROC) assays to define a “methylation score” threshold differentiating cervical cancer from noncancer cervical tissue by using measurements of DNA methylation combinations as set forth in a combination of DNA methylation biomarkers for screening and early detection of cervical cancer, said combination comprises of CGIDs derived using the APDMA method, for detecting cervical cancer by measuring DNA methylation levels of said CGIDs in DNA originating from cervical specimens and deriving a “cervical cancer methylation predictor” using linear regression equations and Receiver operating characteristics (ROC) assays, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70.

In an alternate embodiment, the present invention provides a use of Receiver operating characteristics (ROC) assays to define a “methylation score” threshold differentiating cervical cancer from noncancer cervical tissue by using measurements of DNA methylation combinations as set forth in a combination of DNA methylation biomarkers according to claim 9, said CGIDs are as set forth in SEQ ID NO:3, and SEQ ID NO:31.

In an embodiment, the present invention provides a computer-implemented method for obtaining candidate DNA methylation biomarkers for early detection for cervical cancer diagnosis, the method comprising: providing genome wide DNA methylation data of a multitude of independent genomic CG positions, CGIDs of human genome; processing the genome wide DNA methylation data by normalization and deriving normalized DNA methylation beta values; computing Spearman correlation with the normalized DNA methylation beta values between stages of progression of premalignancy, and untransformed cervical cells; obtaining candidate CGIDs with an analysis of progressive DNA methylation alterations (APDMA) to obtain candidate DNA methylation biomarkers for early detection for cervical cancer diagnosis.

### EXAMPLES

The following examples are given by way of illustration of the present invention and therefore should not be construed to limit the scope of the present invention.

### Example 1: Analysis of Progressive DNA Methylation Alterations (APDMA) Method for Identifying and Obtaining CG Positions (CGIDs) Whose Methylation Level is an Early Predictor of Cervical Cancer

The present invention addresses one of the outstanding challenges in cervical cancer screening which is finding robust biomarkers that provide a highly accurate and sensitive assessment of risk that can guide early intervention and treatment. Common approaches have been using a case-control logistic regression on genome wide DNA methylation data to identify sites that are either more of less methylated in cancer cells versus controls. However, it is well known that many statistically significant DNA methylation alterations in cancer detected by these methods are heterogenous and many evolve late in the progression of cancer and therefore of very limited value in early detection, since they are diluted when the frequency of cancer cells in a specimen is low. Moreover, quantitative differences in methylation profiles rather than categorical differences could be erased in a mixture of normal and cancer cells. As is well understood, DNA methylation is a binary property, which means that a given cell is either methylated or not at a specific CG position in the genome.

In this example, the present invention relates to selected methylated CGIDs as fundamental characteristic of cervical cancer that are almost uniformly methylated across cervical cancer specimens, but are never methylated in normal tissue and despite being categorical for cervical cancer, they emerge very early in the premalignant stages in a milieu of normal cells and progressively increase in frequency from CIN1 to CIN3 stages advancing towards cervical cancer. Methylated CGIDs that are categorically different between normal and cancer tissues have been found to be detected even when cancer cells were found in low frequency in a specimen by deep sequencing of bisulfite converted DNA which provides single DNA molecule resolution. The frequency of molecules with a methylated CGID represents the fraction of cancer cells in the sample. Methylation measurements of such CGIDs by other methods would also determine the incidence of cancer cells in the specimen and is useful as DNA methylation biomarkers for risk and prediction of cervical cancer in the sample.

It is clinically known that a fraction of CIN premalignant lesions develop into cervical cancer, thus they offer a particularly unique window for detecting early DNA methylation alterations in cancer. Predicting early who is going to develop cervical cancer is of utmost clinical significance. The present invention provides a method to obtain such early detection DNA methylation biomarkers characterized by following technical features: first, methylated CGIDs that were categorically characteristic of early cancer cells were uniformly unmethylated in normal cervical tissue; second, these CGIDs were infrequently methylated in early premalignant specimens; third, the frequency of these primary methylated CGIDs should increase with progression of the premalignant stages from CIN1 to CIN3 as predicted by the increased risk of cervical cancer in women with CIN3 lesions; and fourth, since methylation of these CGIDs is a primary characteristic of cervical cancer, these CGIDs should be uniformly abundant in cervical cancer specimens. In this example, specific CGIDs whose methylation increases with progression of CIN stages from CIN1 to CIN3, were found to be ubiquitously methylated in cervical cancer specimens, while they were found to be uniformly unmethylated in normal tissue as delineated herein. Thus, the presently disclosed method of the present invention provides a panel of candidate CGID biomarkers for early detection of cervical cancer in women, particularly those with premalignant lesions.

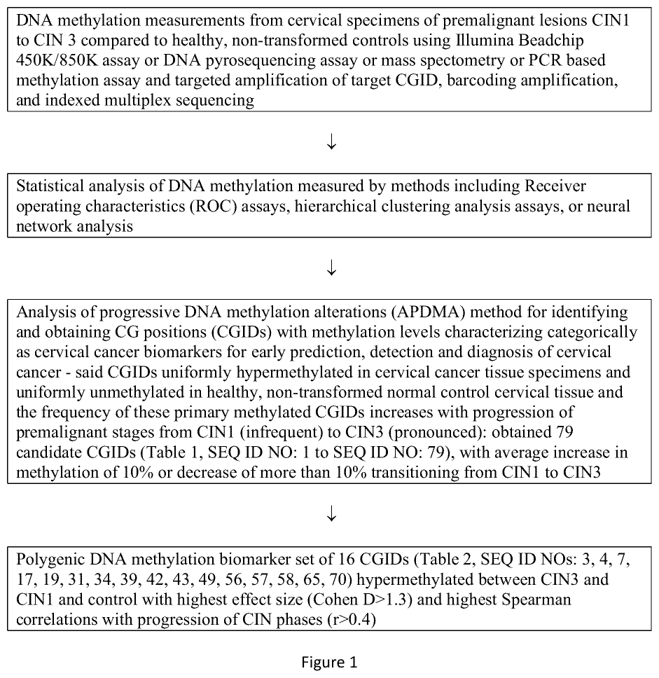

The following steps of the progressive DNA methylation alterations (APDMA) method were performed to delineate CGID biomarkers whose state of methylation detects early cervical cancer as summarized in FIG. 1.

### Cervical Specimens

The present invention used cervical specimens collected from women referred for colposcopic examination in a McGill University affiliated hospital because of an abnormal cervical cancer screening result or for initial treatment of a cervical lesion (19). Briefly, 643 women aged 16-70 years were enrolled between June 2015 and April 2016. Specimens were tested for the presence of HPV DNA of carcinogenic types with the Roche cobas® 4800 HPV Test which detects HPV1 and HPV18 separately, and 12 other high-risk types (HPVs 31, 33, 35, 39, 45, 51, 52, 56, 58, 59, 66, and 68) as a pooled result. Cytology was classified according to the Bethesda classification as NILM: Negative for Intraepithelial Lesion or Malignancy; ASC-US: Atypical Squamous Cells-of Undetermined Significance; ASC-H Atypical Squamous Cells-cannot exclude HSIL; LSIL: Low Squamous Intraepithelial Lesion; HSIL: High Squamous Intraepithelial Lesion; AGC: Atypical Glandular Cells; and cancer (20). Cervical abnormalities were biopsied and histological results were graded by senior McGill pathologists as normal, CIN1, CIN2, CIN3, or invasive cancer. The study received ethical approval by institutional review boards at McGill University and the Jewish General Hospital. Study participants provided written informed consent.

The sample set comprised 186 randomly selected, physician-collected specimens of women. Of these specimens, 50 were CIN1, 40 CIN2, and 42 CIN3 compared to 54 specimens with a normal biopsy result.

### DNA Extraction and Genome Wide Methylation Analysis

DNA was extracted from original exfoliated cervical cell specimens, suspended in liquid-based cytology PreservCyt solution (PreservCyt, Hologic Inc., Mississauga).

Extracted DNA using Qiagen DNA extraction kit was subjected to bisulphite treatment and hybridization to Illumina Epic arrays using standard procedures described by the manufacturer at the Genome Quebec Innovation center in Montreal. Epic arrays provide an excellent coverage of the human promoter and enhancer repertoire, representing all known regions regulating transcription (21).

Normalization and deriving normalized DNA methylation values (beta) for all samples

Samples were randomized with respect to slide and position on arrays and all samples were hybridized and scanned concurrently to mitigate batch effects as recommended by McGill Genome Quebec innovation center according to Illumina Infinium HD technology user guide. Illumina arrays hybridizations and scanning were performed by the McGill Genome Quebec Innovation center according to the manufacturer guidelines. Illumina arrays were analyzed using the ChAMP Bioconductor package in R by Morris et al., 2014 (25). IDAT files were used as input in the champ.load function using minfi quality control and normalization options. Raw data were filtered for probes with a detection value of P>0.01 in at least one sample. The present method filtered out probes on the X or Y chromosome to mitigate sex effects and probes with SNPs as identified in Marzouka et al., 2015 (24), as well as probes that align to multiple locations as identified in Marzouka et al., 2015 (24). Batch effects were analyzed on the non-normalized data using the function champ.svd. Five out of the first 6 principal components were associated with group and batch (slides). Intra-array normalization to adjust the data for bias introduced by the Infinium type 2 probe design was performed using beta-mixture quantile normalization (BMIQ) with function champ.norm(norm=“BMIQ”) (25). Then the batch effects were corrected after BMIQ normalization using champ.runcombat function.

Discovery of CGIDs whose frequency of methylation correlates with CIN progression

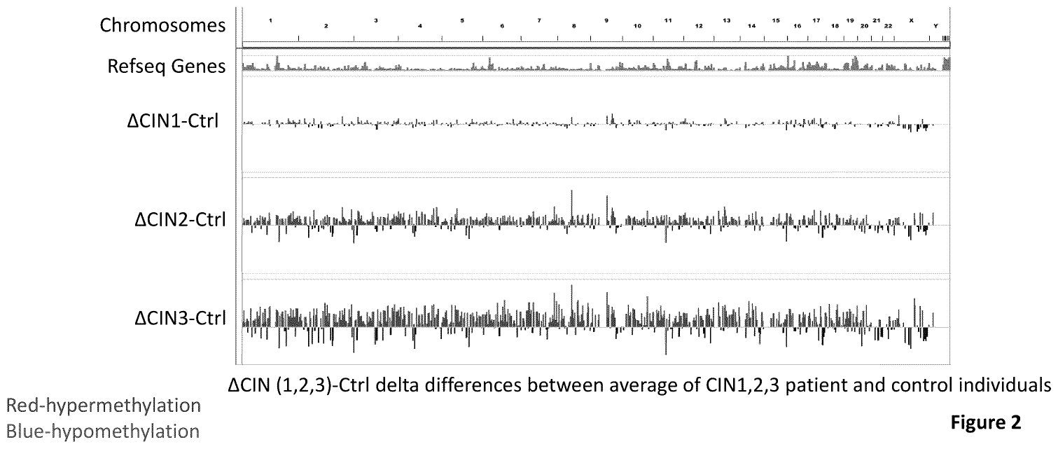

The present method then used the beta-values of the batch corrected normalized data to compute Spearman correlation between CIN stages (with stage codes 0 for non-transformed, healthy control cervical cells and 1-3 for CIN stages from CIN1 to CIN3) using the spearman corr function in R and correcting for multiple testing using the method “fdr” of Benjamini Hochberg (adjusted P value (Q) of <0.05). Methylation levels of 7715 CGIDs significantly correlated (q>0.05) with progression of premalignant CIN stages from 1 to 3 (refer to FIG. 2). Most of the sites were hypermethylated as premalignant lesions progressed from normal to CIN1 to CIN3 stages, while a small fraction was hypomethylated (refer to FIG. 2).

### Shortlisting of Candidate CGIDs

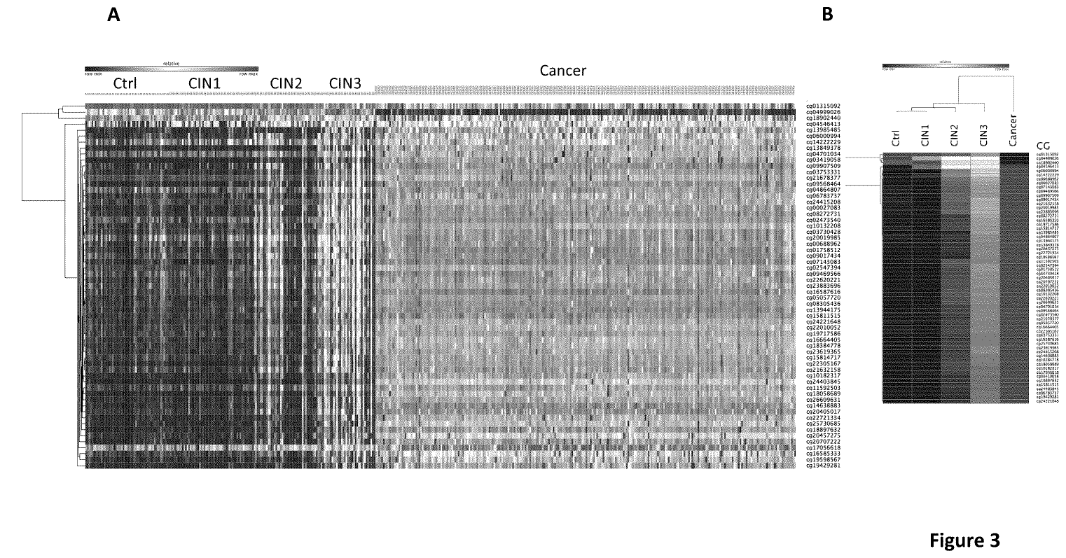

To identify CGID positions that address the assumptions of the APDMA method 79 progressively methylated CGIDs were shortlisted with average increase in methylation of 10% or decrease of more than 10% during transition from CIN1 to CIN3 and background methylation in normal cells (less than 10%) (refer to Table 1 herein above). The present method then tested whether these CGIDs uniformly identify cervical cancer in publicly available Illumina 450K genome wide DNA methylation data from 270 cervical cancer specimens (refer to GSE68339). Based on the tested CGID DNA methylation, the present method then generated a heatmap with these 79 CGIDs whose frequency of methylation increased during progression of cervical premalignant phases that were obtained by the presently disclosed APDMA method. The said heatmap revealed that these 79 CGIDs exhibit a categorically different DNA methylation profile between cervical cancer and normal cervix. The clear majority of sites were totally unmethylated in normal tissue and heavily methylated in cancer tissue while a small number of sites were methylated in normal tissue and unmethylated in cervical cancer (refer to FIG. 3). Thus, the present method relates to these hypermethylated CGIDs as preferred biomarkers since even a low frequency of methylation is clearly detectable on a background of totally unmethylated molecules.

### Example 2: Discovery of a Polygenic DNA Methylation Biomarker Set for Early Detection of Cervical Cancer

The present disclosure further shortlists 16 CGIDs from the list obtained and disclosed in the first example and in Table 1, where the said 16 CGIDs were hypermethylated between CIN3 and CIN1 and control, had the highest effect size (Cohen D>1.3) between CIN3 and control and highest Spearman correlation with progression of CIN phases r>0.4. (refer to Table 2 herein above).

Next, in order to obtain the minimal number of CGIDs required for differentiating CIN3 premalignant lesions from control, the present method performed a penalized regression which reduced the number of CGIDs to 5. The present method then performed a multivariable linear regression with these 5 CGIDs as independent variables and CIN3 state as the dependent variable. Two CGIDs remained significant (refer to Table 3 herein above). A linear regression equation composed of weighted methylation levels of these two sites was highly significant for prediction of CIN3 (p<5×10⁻¹⁵).

### Example 3: Utility of Bi-Genic DNA Methylation Markers for Detecting Cervical Cancer

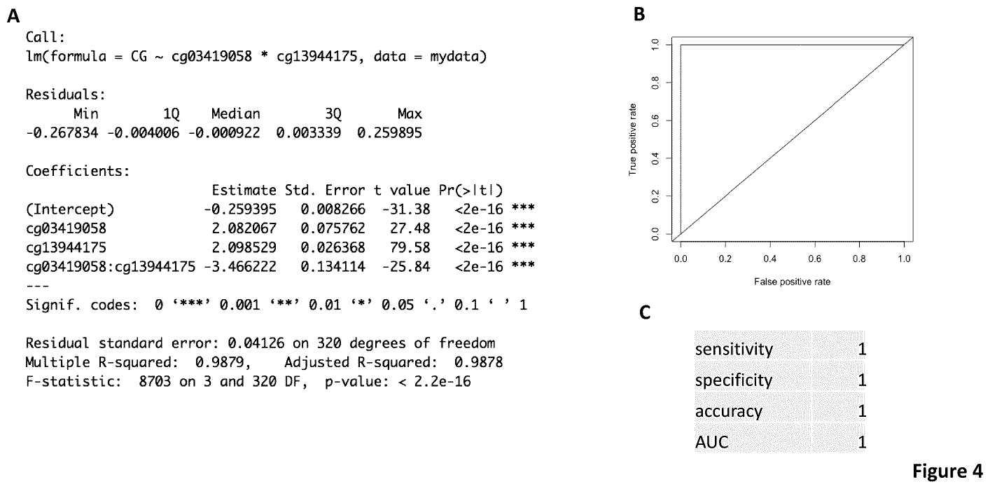

Next, the present disclosure first validated the bigenic DNA methylation marker (cg03419058; cg13944175) on the publicly available data base of cervical cancer 450K DNA methylation (refer to GSE68339). A bivariable linear regression model with cervical cancer as the dependent variable and the level of methylation of the two CGIDs (cg03419058; cg13944175) as independent variables was observed to be highly significant (p<2.2×10⁻¹⁶, F=8703, R=0.9873). ROCs for the methylation scores (calculated using the linear regression equation as disclosed in FIG. 4A) were compared by calculating their area under the curve (AUC) (refer to FIG. 4B). The sensitivity and specificity of the bigenic methylation score for discriminating cervical cancer from normal cervical tissue was observed to be 1 (refer to FIG. 4C).

Thus, the above disclosed DNA methylation biomarkers and the calculated methylation score are useful for screening and early detection of cervical cancer in women at risk as well as the general healthy population of women using cervical specimens collected at routine gynecological checkup pap smears.

### Example 4: Utility of Bi-Genic DNA Methylation Biomarkers for Measuring Cervical Cancer Methylation Scores in Individual Specimens from Healthy Controls, CIN1 to CIN3 and Cervical Cancer Patients

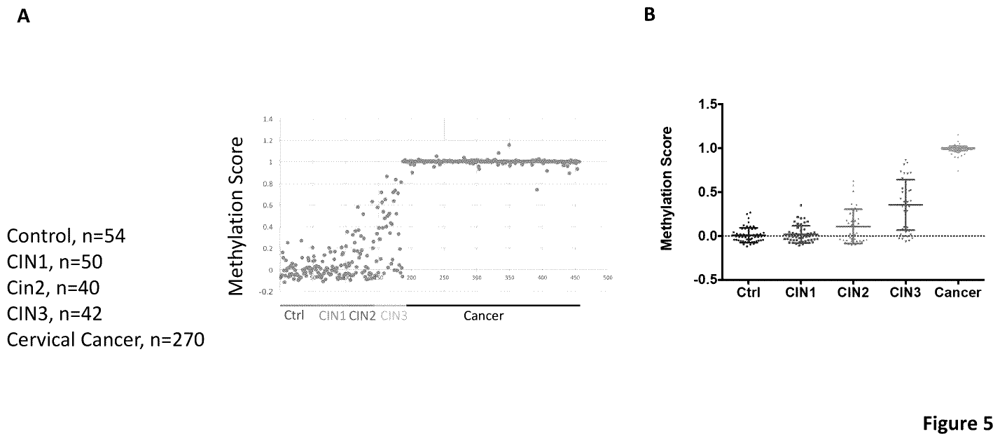

Methylation scores (cervical cancer prediction) were calculated using the equation presented in FIG. 4A for each of the individual specimens from controls, CIN1 to CIN3 (from the McGill cohort described herein above in Example 1) and cervical cancer (refer to GSE68339) (refer to FIG. 5A), (for mean values for the different groups, refer to FIG. 5B). The results illustrate increase in methylation scores in advanced premalignant lesions as anticipated from the clinical observation of increased risk for cervical cancer with progression of CIN stages. Methylation scores could be used for screening of women with CIN lesions for risk of cervical cancer.

### Example 5: Spearman Correlation of Methylation Score and Progression of Premalignant Cervical Cancer to Cervical Cancer

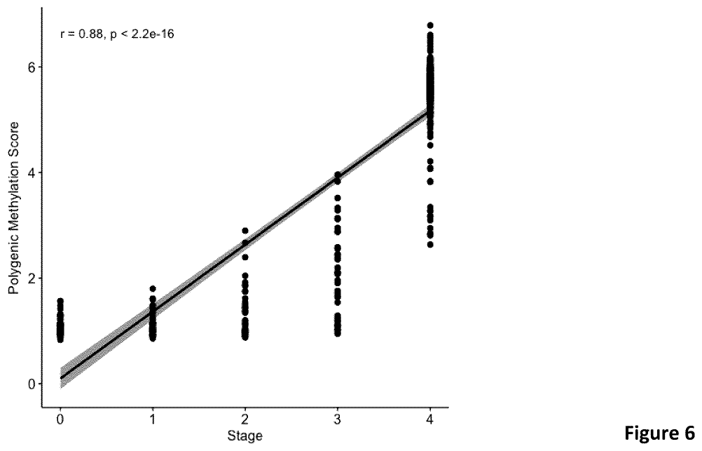

A Spearman correlation analysis was performed between methylation scores of cervical specimens from healthy, premalignant stages CIN1 to CIN3 and cervical cancer (Control, n=54; CIN1, n=50; CIN2, n=40; CIN3, n=42; Cervical Cancer, n=270). The results illustrate highly significant correlation (p<2.2×10⁻¹⁶ and r=0.88) between methylation score of bi-genic marker and progression from premalignancy to malignancy (refer to FIG. 6).

### Example 6: Validation of Methylation Biomarker (cg13944175) for Detecting Cervical Cancer

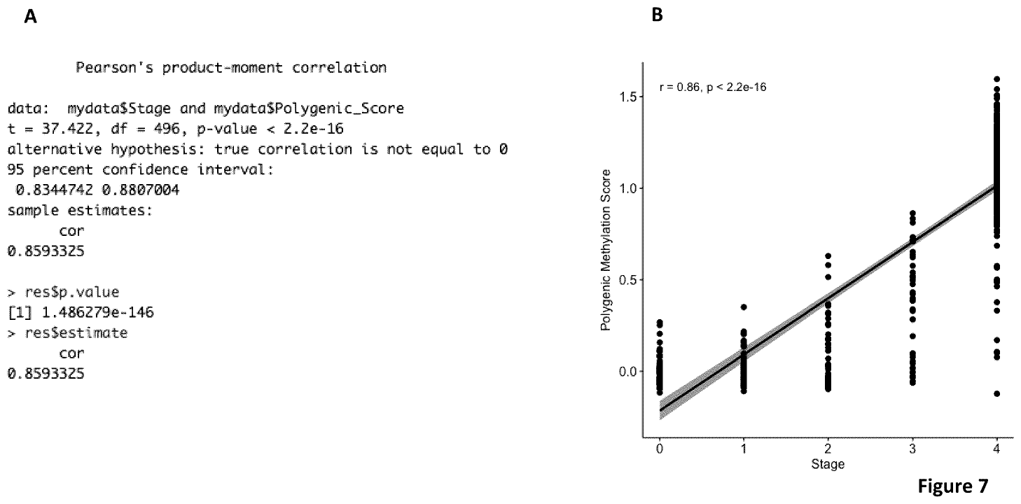

Since data for only one CGID biomarker was available in TCGA cervical cancer data, the present disclosure calculated the methylation score for cervical cancer using a linear regression equation with DNA methylation data for only said CGID, cg13944175. A Spearman correlation was calculated between stage of progression to cancer and the methylation score (refer to statistics in FIG. 7A and correlation chart in FIG. 7B). In this disclosure, CIN1 to CIN3 are from the McGill cohort as have already been described in this application in Example 1 and the assignment of the score is based on the assigned Scale: Control: 0, CIN1-3:1-3, respectively, and cervical cancer: 4.

### Example 7. Utility of Bi-Genic Methylation Biomarker for Detecting Cervical Cancer in Premalignant Cervical Specimens

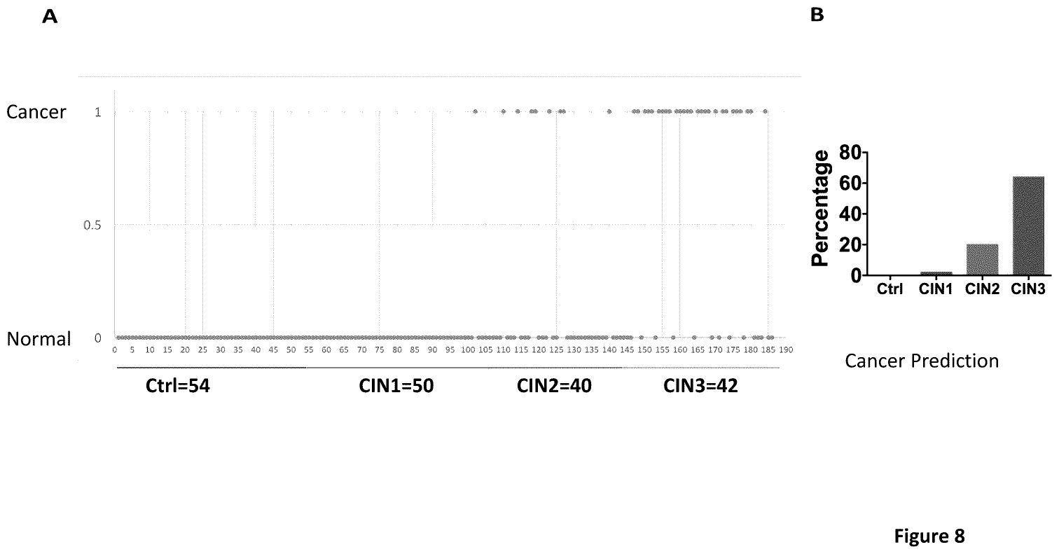

The bi-genic methylation biomarker was used to predict which of the CIN1 to CIN3 samples will progress to cervical cancer. Methylation scores were calculated for each specimen based on the methylation values for the two CG sites obtained from epic Arrays data. Using the threshold for cancer computed from comparisons of cervical cancer and healthy cervical specimens (refer to FIG. 3) a prediction was made for each of the samples (refer to FIG. 8A). The fraction of specimens that were predicted to become cancerous increased from a few in CIN1 specimens to 60% of the CIN3 specimens as expected (refer to FIG. 8B).

Although the invention has been explained in relation to its preferred embodiment, it is to be understood that many other possible modifications and variations can be made without departing from the spirit and scope of the invention.

### Advantages

These novel DNA methylation biomarkers could be developed as a diagnostic kit for early and accurate diagnosis of human cervical cancer. They are direct indicators of cellular changes during the initiation and development of cervical cancer and present a fundamental characteristic of cervical cancer that are almost uniformly methylated across cervical cancer specimens, but are never methylated in normal tissue and progressively increase in frequency from CIN1 to CIN3 premalignant stages. These biomarkers complement pathology for accurate early detection of cervical cancer in CIN lesions as well as serve as early detection and as a risk prediction biomarker in asymptomatic women. These biomarkers provided a utility angle to the already existing epigenetic, DNA methylation markers which play a major role in gene regulation, usable in form of CGIDs as a tool of diagnosis. These biomarkers could provide a fast, cheaper, accurate, robust and high throughput diagnostic kit for accurate, early and as yet unfeasible diagnosis of human cervical cancer at as yet inaccessible premalignant stages.

### References

El-Zein M, Richardson L, Franco E L. Cervical cancer screening of HPV vaccinated populations: Cytology, molecular testing, both or none. J. Clin. Virol. 2015;76: S62-S68. doi:10.1016/j.jcv.2015.11.020.
Boers A, Wang R, van Leeuwen R W, et al. Discovery of new methylation markers to improve screening for cervical intraepithelial neoplasia grade 2/3. Clin. Epigenetics 2016; 8(29). doi:10.1186/s13148-016-0196-3.
van Baars R, van der Marel J, Snijders P J F, et al. CADM1 and MAL methylation status in cervical scrapes is representative of the most severe underlying lesion in women with multiple cervical biopsies. Int. J. Cancer 2016; 138:463-471. doi:10.1002/ijc.29706.
Verhoef V M J, Van Kemenade F J, Rozendaal L, et al. Follow-up of high-risk HPV positive women by combined cytology and bi-marker CADM1/MAL methylation analysis on cervical scrapes. Gynecol. Oncol. 2015; 137(1):55-59. doi:10.1016/j.ygyno.2015.01.550.
Louvanto K, Franco E L, Ramanakumar A V, et al. Methylation of viral and host genes and severity of cervical lesions associated with human papillomavirus type 16. Int. J. Cancer 2014; 00(6):1-8. doi:10.1002/ijc.29196.
Simanaviciene V, Popendikyte V, Gudleviciene Z, Zvirbliene A. Different DNA methylation pattern of HPV16, HPV18 and HPV51 genomes in asymptomatic HPV infection as compared to cervical neoplasia. Virology 2015; 484:227-233. doi:10.1016/j.virol.2015.06.008.
De Strooper L M A, Meijer C J L M, Berkhof J, et al. Methylation analysis of the FAM19A4 gene in cervical scrapes is highly efficient in detecting cervical carcinomas and advanced CIN2/3 lesions. Cancer Prev. Res. 2014; 7(6):1251-1257. doi:10.1158/1940-6207.CAPR-14-0237.
Mersakova S, Nachajova M, Szepe P, Kasajova P S, Halasova E. DNA methylation and detection of cervical cancer and precancerous lesions using molecular methods. Tumor Biol. 2015. doi:10.1007/s13277-015-4197-1.
Nikolaidis C, Nena E, Panagopoulou M, et al. PAX1 methylation as an auxiliary biomarker for cervical cancer screening: a meta-analysis. Cancer Epidemiol. 2015; 39(5):682-686. doi:10.1016/j.canep.2015.07.008.
De Strooper L M A, Hesselink A T, Berkhof J, et al. Combined CADM1/MAL methylation and cytology testing for colposcopy triage of high-risk HPV-positive women. Cancer Epidemiol. Biomarkers Prev. 2014; 23(9):1933-1937. doi:10.1158/1055-9965.EPI-14-0347.
De Strooper L M A, Verhoef V M J, Berkhof J, et al. Validation of the FAM19A4/mir124-2 DNA methylation test for both lavage- and brush-based self-samples to detect cervical (pre)cancer in HPV-positive women. Gynecol. Oncol. 2016. doi:10.1016/j.ygyno.2016.02.012.
Louvanto K, Franco E L, Ramanakumar A V, et al. Methylation of viral and host genes and severity of cervical lesions associated with human papillomavirus type 16. Int. J. cancer 2015; 136(6):E638-45. doi:10.1002/ijc.29196.
De Strooper LM a, van Zummeren M, Steenbergen R D M, et al. CADM1, MAL and miR124-2 methylation analysis in cervical scrapes to detect cervical and endometrial cancer. J. Clin. Pathol. 2014; 67:1067-1071. doi:10.1136/jclinpath-2014-202616.
Feng C, Dong J, Chang W, Cui M, Xu T. The Progress of Methylation Regulation in Gene Expression of Cervical Cancer. Int. J. Genomics 2018; 2018.
Del Mistro A, Frayle H, Rizzi M, et al. Methylation analysis and HPV genotyping of self-collected cervical samples from women not responding to screening invitation and review of the literature. PLoS One 2017; 12(3):1-13. doi:10.1371/journal.pone.0172226.
Eijsink J J H, Lendvai A, Deregowski V, et al. A four-gene methylation marker panel as triage test in high-risk human papillomavirus positive patients. Int. J. Cancer 2012; 130(8):1861-1869. doi:10.1002/ijc.26326.
Verlaat W, Snoek B C, Heideman D A M, et al. Identification and validation of a 3-gene methylation classifier for HPV-based cervical screening on self-samples. Clin. Cancer Res. 2018:clincanres.3615.2017. doi:10.1158/1078-0432.CCR-17-3615.
Cuzick J, Bergeron C, von Knebel Doeberitz M, et al. New technologies and procedures for cervical cancer screening. Vaccine 2012; 30(SUPPL.5):F107-F116. doi:10.1016/j.vaccine.2012.05.088.
El-Zein M, Bouten S, Louvanto K, et al. Validation of a new HPV self-sampling device for cervical cancer screening: The Cervical and Self-Sample In Screening (CASSIS) study. Gynecol. Oncol. 2018. doi:https://doi.org/10.1016/j.ygyno.2018.04.004.
Smith J H F. Bethesda 2001. Cytopathology 2002; 13(1):4-10.
Moran S, Arribas C, Esteller M. Validation of a DNA methylation microarray for 850,000 CpG sites of the human genome enriched in enhancer sequences. Epigenomics 2016; 8(3):389-399. doi:10.2217/epi.15.114.
Morris T J, Butcher L M, Feber A, et al. ChAMP: 450k Chip Analysis Methylation Pipeline. Bioinformatics 2014; 30(3):428-430. doi:10.1093/bioinformatics/btt684.
Luttmer R, De Strooper L M A, Berkhof J, et al. Comparing the performance of FAM19A4 methylation analysis, cytology and HPV16/18 genotyping for the detection of cervical (pre)cancer in high-risk HPV-positive women of a gynecologic outpatient population (COMETH study). Int. J. Cancer 2015; 138(May 2015):992-1002. doi:10.1002/ijc.29824.
Wentzensen N, Schiffman M, Palmer T, Arbyn M. Triage of HPV positive women in cervical cancer screening. J. Clin. Virol. 2016; 76:S49-S55. doi:10.1016/j.jcv.2015.11.015.
ADDIN EN.REFLIST Marzouka, N. A., Nordlund, J., Backlin, C. L., Lonnerholm, G., Syvanen, A. C., & Carlsson Almlof, J. (2015). CopyNumber450kCancer: baseline correction for accurate copy number calling from the 450k methylation array. Bioinformatics. doi:10.1093/bioinformatics/btv652
Morris, T. J., Butcher, L. M., Feber, A., Teschendorff, A. E., Chakravarthy, A. R., Wojdacz T. K., & Beck, S. (2014). ChAMP: 450k Chip Analysis Methylation Pipeline. Bioinformatics, 30(3), 428-430. doi:10.1093/bioinformatics/btt684

What is claimed is:

1. An in-vitro method for obtaining early predictors of cervical cancer, the method comprising the steps of: (a) measuring DNA methylation from a cervical specimen sample, (b) performing statistical analysis on the DNA methylation measurement obtained in step a, (c) determining DNA methylation status of a multitude of independent genomic CG positions called CG identifiers (CGIDs) by performing analysis of progressive DNA methylation alterations (APDMA) of genome wide DNA methylation profiles obtained in step b, (d) classifying CGIDs based on frequency of their DNA methylation correlating with cervical cancer premalignant stage progression, (e) obtaining candidate CGIDs from classification in step d to obtain early predictors of cervical cancer as DNA methylation biomarkers, wherein said candidate CGIDs as the early predictors of cervical cancer as DNA methylation biomarkers, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70 and combinations thereof.

2. The method according to claim 1, said measuring DNA methylation is performed using methods comprising, Illumina 27K, 450K or 850K arrays, genome wide bisulfite sequencing on platforms including, HiSeq, MiniSeq, MiSeq or NextSeq sequencers, torrent sequencing, methylated DNA Immunoprecipitation (MeDIP) sequencing, hybridization with oligonucleotide arrays, DNA pyrosequencing, mass spectrometry based (Epityper™) or PCR based methylation assays; and said statistical analysis on the DNA methylation measurement includes Pearson correlation, Receiver operating characteristics (ROC) assays, and hierarchical clustering analysis.

3. The method according to claim 1, said cervical cancer premalignant stage progression comprises cervical intraepithelial neoplasia lesions at stages CIN1, CIN2 and CIN3.

4. A use of multivariable linear regression equation or neural network analysis for calculating a methylation score predicting cervical cancer by using measurements of DNA methylation CGIDs and combinations thereof according to the method of any one of the claims 1 to 3.

5. A use of Receiver operating characteristics (ROC) assays to define a “methylation score” threshold differentiating cervical cancer from noncancer cervical tissue by using measurements of DNA methylation and combinations thereof according to the method of any one of the claims 1 to 3.

6. A computer-implemented method for obtaining candidate DNA methylation biomarkers for early detection for cervical cancer diagnosis, the method comprising: providing genome wide DNA methylation data of a multitude of independent genomic CG positions, CGIDs of human genome; processing the genome wide DNA methylation data by normalization and deriving normalized DNA methylation beta values; computing Spearman correlation with the normalized DNA methylation beta values between stages of progression of premalignancy, and untransformed cervical cells; obtaining candidate CGIDs with an analysis of progressive DNA methylation alterations (APDMA) to obtain candidate DNA methylation biomarkers for early detection for cervical cancer diagnosis, wherein said candidate CGIDs as the early predictors of cervical cancer as DNA methylation biomarkers, wherein the CGIDs are selected from a group as set forth in SEQ ID NO:3, SEQ NO:4, SEQ ID NO:7, SEQ ID NO:17, SEQ ID NO:19, SEQ ID NO:31, SEQ ID NO:34, SEQ ID NO:39, SEQ ID NO:42, SEQ ID NO:43, SEQ ID NO:49, SEQ ID NO:56, SEQ ID NO:57, SEQ ID NO:58, SEQ ID NO:65, and SEQ ID NO:70 and combinations thereof.


SEQUENCE LISTING (91 sequences)

SEQ ID NO: 1 - DNA, 122 bp, synthetic construct
  misc_feature 1..122: note = Description of Artificial Sequence: Synthetic polynucleotide
  source 1..122: mol_type = other DNA
gaaggaggctgcgcgccagcccgcccgcggcgcccgggctcaggcgccgtgacggctgcacgcgctgccccgcactctgagggccttcattagctcgctccccgcgccgaggctggggcggg

SEQ ID NO: 2 - DNA, 122 bp, synthetic construct
  misc_feature 1..122: note = Description of Artificial Sequence: Synthetic polynucleotide
  source 1..122: mol_type = other DNA
cctcccgcagctcattgcagccccgaggaaatcaccgggggagggctcgggagtgcggcgcggcagccccataatttccagggcccttctcctacactgacacgtaattgtcagattgtttt

SEQ ID NO: 3 - DNA, 122 bp, synthetic construct
  misc_feature 1..122: note = Description of Artificial Sequence: Synthetic polynucleotide
  source 1..122: mol_type = other DNA
ccgccgcgggttcccagggctggtggtagttgccgtcccacacgtacgtggcggggtcctcgtcagcgaagacctcgcggaacatgtcgaccatgtagaggtcctcggcgcggttgccatcc

SEQ ID NO: 4 - DNA, 122 bp, synthetic construct
  misc_feature 1..122: note = Description of Artificial Sequence: Synthetic polynucleotide
  source 1..122: mol_type = other DNA
ggggaggaatattagactcggaggagtctgcgcgcttttctcctccccgcgcctcccggtcgccgcgggttcaccgctcagtccccgcgctcgctccgcaccccacccacttcctgtgctcg

SEQ ID NO: 5 - DNA, 122 bp, synthetic construct
  misc_feature 1..122: note = Description of Artificial Sequence: Synthetic polynucleotide
  source 1..122: mol_type = other DNA
caggccggtcccagccgcccggagccccagtgcgcgatggcggccggcaaactgcgcctgcgcactgggcctcaccgcggactacgactcccacaatgccgcgaggctgtgccgcgcaccgg

SEQ ID NO: 6 - DNA, 122 bp, synthetic construct
  misc_feature 1..122: note = Description of Artificial Sequence: Synthetic polynucleotide
  source 1..122: mol_type = other DNA
gtgacgcgcggccgcagctgcccgcgggcggagcgctctcagaccccggagcgcacaccgcggggccatcggtgccatcgcggatctccaggctcctcatcagtccgccggggccgcagcag

SEQ ID NO: 7 - DNA, 122 bp, synthetic construct
  misc_feature 1..122: note = Description of Artificial Sequence: Synthetic polynucleotide
  source 1..122: mol_type = other DNA
gaggaatattagactcggaggagtctgcgcgcttttctcctccccgcgcctcccggtcgccgcgggttcaccgctcagtccccgcgctcgctccgcaccccacccacttcctgtgctcgccc

SEQ ID NO: 8 - DNA, 122 bp, synthetic construct
  misc_feature 1..122: note = Description of Artificial Sequence: Synthetic polynucleotide
  source 1..122: mol_type = other DNA
atctaccgtctccaatctccatctccgaagttatgcccacttcctcgaagtttggagccacgcgaactacactgcccagaaggcgccgcgccgtgagccgcgatgcttggccaatgaaaaga

SEQ ID NO: 9 - DNA, 122 bp, synthetic construct
  misc_feature 1..122: note = Description of Artificial Sequence: Synthetic polynucleotide
  source 1..122: mol_type = other DNA
gggagggctcgtgagagccaatgagagcgcggaaggcggcgagcgagccaatggacgcggcggtggggcagggggcggggcctgggcgaggccgggggcggaatgggctgagtgccctgtct

SEQ ID NO: 10 - DNA, 122 bp, synthetic construct
  misc_feature 1..122: note = Description of Artificial Sequence: Synthetic polynucleotide
  source 1..122: mol_type = other DNA
cggcaagcggagcagcgaggcagggtagcttcatcacactcgcggcggatgcggattccgcgccgccccggctctagctgctcaggcgaccgccaccctcgcctcgccgccgcccgtgcaca

SEQ ID NO: 11 - DNA, 122 bp, synthetic construct
  misc_feature 1..122: note = Description of Artificial Sequence: Synthetic polynucleotide
  source 1..122: mol_type = other DNA
gcggacggcggctccatccgcggcaatcaccgtagtgcttgtttgtggaagccgagcgtgcgtgcgccgcgcgcgcacccagtccagcgcggagtgggcgtctacccgaggaggggtgtctg

SEQ ID NO: 12 - DNA, 122 bp, synthetic construct
  misc_feature 1..122: note = Description of Artificial Sequence: Synthetic polynucleotide
  source 1..122: mol_type = other DNA
tggggaattagctcaggcggtggagcgctcgcttagctatgcgagaggtagcgagatcgacgcccgcattctccagtttcttgtctggtttatgtctcttagtttgtattccccgttgtttc

SEQ ID NO: 13 - DNA, 122 bp, synthetic construct
  misc_feature 1..122: note = Description of Artificial Sequence: Synthetic polynucleotide
  source 1..122: mol_type = other DNA
gaagtcccagggacctgcggagcgcagacataacacaacacagagcaaaactcaccgctgcggtgactttcactccacgcgatccgcttcccggtttacgctaaactgggcgctcgggacag

SEQ ID NO: 14 - DNA, 122 bp, synthetic construct
  misc_feature 1..122: note = Description of Artificial Sequence: Synthetic polynucleotide
  source 1..122: mol_type = other DNA
ggctgcggacggcggctccatccgcggcaatcaccgtagtgcttgtttgtggaagccgagcgtgcgtgcgccgcgcgcgcacccagtccagcgcggagtgggcgtctacccgaggaggggtg

SEQ ID NO: 15 - DNA, 122 bp, synthetic construct
  misc_feature 1..122: note = Description of Artificial Sequence: Synthetic polynucleotide
  source 1..122: mol_type = other DNA
cccccgccggccgccggccgcgctccccgccttcattctgtgatctgcggatttgccagtcgccaacctccgcgcccagagtcaccatcgcgcagggttgggcaaaccatggagctcggggc

SEQ ID NO: 16 - DNA, 122 bp, synthetic construct
  misc_feature 1..122: note = Description of Artificial Sequence: Synthetic polynucleotide
  source 1..122: mol_type = other DNA
aactcctgcacaaatcatttcaaacgcggtcggcttctaatcgggaagtaatctcagtgacgctggcggtgcagagaaccgagtctggacgcacacacacaaacacaccgcgggcctccgca

SEQ ID NO: 17 - DNA, 122 bp, synthetic construct
  misc_feature 1..122: note = Description of Artificial Sequence: Synthetic polynucleotide
  source 1..122: mol_type = other DNA
gtgtgctcagcctcagcgtgaggggcacctgctcgtctgggctcacagcgaaggcagcctcgccgcgagctgccgctgccgctgctgccgccactggtgttgccgctctcaggcgccaggct

SEQ ID NO: 18 - DNA, 122 bp, synthetic construct
  misc_feature 1..122: note = Description of Artificial Sequence: Synthetic polynucleotide
  source 1..122: mol_type = other DNA
gccgggagcctgacgtcaccacgccctgcctgtcaatctgcagcgcgcgccgctcgcagccgccttttctgccaccaactgtatctctcactcgcggagccggcacagcgacaggcgccccg

SEQ ID NO: 19 - DNA, 122 bp, synthetic construct
  misc_feature 1..122: note = Description of Artificial Sequence: Synthetic polynucleotide
  source 1..122: mol_type = other DNA
gcggcggcgggcggggagccaggcccgagctgcgttctgcgcagccattggtgggcgccgcgctctgcactgagcatgttcgcgccccgccggcccctagccgcagccgcagccgcagcgac

SEQ ID NO: 20 - DNA, 122 bp, synthetic construct
  misc_feature 1..122: note = Description of Artificial Sequence: Synthetic polynucleotide
  source 1..122: mol_type = other DNA
caaccggttccgccgcgtttgtgggctggtagcccggaatacatttcccagaggccttcgcggccgacgtgcttcgcgcaggaacgcagccgcctcccgactggaggacgcggtagcggagc

SEQ ID NO: 21 - DNA, 122 bp, synthetic construct
  misc_feature 1..122: note = Description of Artificial Sequence: Synthetic polynucleotide
  source 1..122: mol_type = other DNA
gctgcccgtggtcaaactggagtcgctgaagcgctggaacgaagagcggggcctctggtgcgagaagggggtgcaggtgctgctgacgacggtgggcgccttcgccgccttcggcctcatga

SEQ ID NO: 22 - DNA, 122 bp, synthetic construct
  misc_feature 1..122: note = Description of Artificial Sequence: Synthetic polynucleotide
  source 1..122: mol_type = other DNA
cttcccggctccccgcggtgcgcacccgctggccactctgcgcacgcgcgccgggtgccccggcctaaggccgttgacctcgggttctccccggcacagtcgaatccacgccagggccctca

SEQ ID NO: 23 - DNA, 122 bp, synthetic construct
  misc_feature 1..122: note = Description of Artificial Sequence: Synthetic polynucleotide
  source 1..122: mol_type = other DNA
gcgggggaggttgcgggggaggctcggcgtccccgctctccgccccgcgacaccgactgccgccgtggccgccctcaaagctcatggttgtgccgccgccgccctcctgccggcccggctgg

SEQ ID NO: 24 - DNA, 122 bp, synthetic construct
  misc_feature 1..122: note = Description of Artificial Sequence: Synthetic polynucleotide
  source 1..122: mol_type = other DNA
tgtactacttcctctgccacctggccttggtagacgcgggcttcactactagcgtggtgccgccgctgctggccaacctgcgcggaccagcgctctggctgccgcgcagccactgcacggcc

SEQ ID NO: 25 - DNA, 122 bp, synthetic construct
  misc_feature 1..122: note = Description of Artificial Sequence: Synthetic polynucleotide
  source 1..122: mol_type = other DNA
aaaaaaaaaaaaaagcaatgagccgcaagccttggactcgcagagctgccggtgcccgtccgagagccccaccagcgcggctcacgcctcagtctcgccgccccaaggtgggatccgacgcc

SEQ ID NO: 26 - DNA, 122 bp, synthetic construct
  misc_feature 1..122: note = Description of Artificial Sequence: Synthetic polynucleotide
  source 1..122: mol_type = other DNA
cgagagggcccggtccagcagcctctggggcccagtgcgcagggcactgcgggccgattgcgccccggggccaggaggcgccgagaaagcaaaagcaaaagccggcggcgggtggaggtcaa

SEQ ID NO: 27 - DNA, 122 bp, synthetic construct
  misc_feature 1..122: note = Description of Artificial Sequence: Synthetic polynucleotide
  source 1..122: mol_type = other DNA
cggccgcagtgtgccgcccgctgcgctatgcggggctcgtctccccgcgcctatgtcgcacgctggccagcgcctcctggctaagcggcctcaccaactcggttgcgcaaaccgcgctcctg

SEQ ID NO: 28 - DNA, 122 bp, synthetic construct
  misc_feature 1..122: note = Description of Artificial Sequence: Synthetic polynucleotide
  source 1..122: mol_type = other DNA
cctggcgcgaccgccagcagcacccagcgcggggccgggagctgctgggggcccaggctccgctctccccaccgctctgcaccgctgccggctgcggacagacccgatgcgccaccaccacc

SEQ ID NO: 29 - DNA, 122 bp, synthetic construct
  misc_feature 1..122: note = Description of Artificial Sequence: Synthetic polynucleotide
  source 1..122: mol_type = other DNA
ccggagcgcgctgctgccctctaccggtcatccgtgcggccggacaccgtgtcaggcccgcgaggagggctctgccgcagtcccggggaacagcacccagcagcgccactgggagaggaaac

SEQ ID NO: 30 - DNA, 122 bp, synthetic construct
  misc_feature 1..122: note = Description of Artificial Sequence: Synthetic polynucleotide
  source 1..122: mol_type = other DNA
agtccagagcggcgctgtgcagctggaagggcgcgcgatagctcaagttagaggcggccccggggcgcggcgcaggacacaagacctcaaactggtacttgcacaggtagccgttggcgcgc

SEQ ID NO: 31 - DNA, 122 bp, synthetic construct
  misc_feature 1..122: note = Description of Artificial Sequence: Synthetic polynucleotide
  source 1..122: mol_type = other DNA
ggcggtgcgagctccccgcctgcgggacgcacggagaccgcggtcagcgcgccgcctggccggcccagcgcgcccagcccgcgcccagccccgtccactcccgtccagccccgccgcccggc

SEQ ID NO: 32 - DNA, 122 bp, synthetic construct
  misc_feature 1..122: note = Description of Artificial Sequence: Synthetic polynucleotide
  source 1..122: mol_type = other DNA
cggtagagtttccaacacgaaagcccgtgtggtcgcgccgggagctcacggcgttccaagcggcacttatcccgcgttgatgcccaggcaccccgcgcgccctgtttcaccaggcccagtca

SEQ ID NO: 33 - DNA, 122 bp, synthetic construct
  misc_feature 1..122: note = Description of Artificial Sequence: Synthetic polynucleotide
  source 1..122: mol_type = other DNA
ccagcggcagtagctgtagcagcttcagcgaagccggagatgggcagagagcgcgcgcggcgcagcagctccagattcactgctctcccctgcagctccccgcgcccccgccgctgtcgctg

SEQ ID NO: 34 - DNA, 122 bp, synthetic construct
  misc_feature 1..122: note = Description of Artificial Sequence: Synthetic polynucleotide
  source 1..122: mol_type = other DNA
gtgttctctgcggcgggccgcgtccccgctgagcctcgcggtgacagccgcctttggcagcgagcgctcggggcacttctatccccgcctctcaaagggtggggacagccgtttccagattt

SEQ ID NO: 35 - DNA, 122 bp, synthetic construct
  misc_feature 1..122: note = Description of Artificial Sequence: Synthetic polynucleotide
  source 1..122: mol_type = other DNA
cggccgcgcccccggcagcccagggcgcgcttccaccacggtaccggtggattcgccgtgcgcagccggaagatggcgcagacgcacaaagcacaccgatgctgcgccatgatagggccggc

SEQ ID NO: 36 - DNA, 122 bp, synthetic construct
  misc_feature 1..122: note = Description of Artificial Sequence: Synthetic polynucleotide
  source 1..122: mol_type = other DNA
tctcgcggcgcaggcggcggcggcagaggtggggtcgcgcagcggaggcagctcgagcttcgggatgcgcgctcgcttcttgggctcctcgctcgatcttactgcccccttttttctctccc

SEQ ID NO: 37 - DNA, 122 bp, synthetic construct
  misc_feature 1..122: note = Description of Artificial Sequence: Synthetic polynucleotide
  source 1..122: mol_type = other DNA
tcctccagccagagtcggtgggactggctgcgctgccctgaagtggttctccaagcagcgcggagggtggcggacggcggacggagcccaggggccgcgtcgggtggggaaacccgaactcg

SEQ ID NO: 38 - DNA, 122 bp, synthetic construct
  misc_feature 1..122: note = Description of Artificial Sequence: Synthetic polynucleotide
  source 1..122: mol_type = other DNA
tgcgcatcgctggctctgggttccgccgaatgcgtcctcctggcggtgatggctctggaccgcgcggccgcagtgtgccgcccgctgcgctatgcggggctcgtctccccgcgcctatgtcg

SEQ ID NO: 39 - DNA, 122 bp, synthetic construct
  misc_feature 1..122: note = Description of Artificial Sequence: Synthetic polynucleotide
  source 1..122: mol_type = other DNA
aggggagctgcgaggcgaagtgttcttcagggaagcgggctcgagtctccgcagctgcggcggcggcggcggcgcgctgggccggcggcgggcgcgggcagggggccgggggtgccgcgcgg

SEQ ID NO: 40 - DNA, 122 bp, synthetic construct
  misc_feature 1..122: note = Description of Artificial Sequence: Synthetic polynucleotide
  source 1..122: mol_type = other DNA
cctccacccccggggggttcctgcgcactgaaagaccgttctccggcaggttttgggatccggcgacggctgaccgcgcgccgcccccacgcccggttccacgatgctgcaatacagaaagt

SEQ ID NO: 41 - DNA, 122 bp, synthetic construct
  misc_feature 1..122: note = Description of Artificial Sequence: Synthetic polynucleotide
  source 1..122: mol_type = other DNA
agagaggggtcccagaacgaaggtggcggcacgagctctgcgctggcggctgtggggggccggcgctcaggaccccaactccatccaagttgcgccgcggtgggggcgggcggaggcggcgc

SEQ ID NO: 42 - DNA, 122 bp, synthetic construct
  misc_feature 1..122: note = Description of Artificial Sequence: Synthetic polynucleotide
  source 1..122: mol_type = other DNA
aatctcccctcgggctcgacggatgtgcgccccagatgtgctgacacatgtccgatgcctcgctgccttggaggtctccccgctcgcgtgtctcttctcttcgcaccagcggcggaaaccgc

SEQ ID NO: 43 - DNA, 122 bp, synthetic construct
  misc_feature 1..122: note = Description of Artificial Sequence: Synthetic polynucleotide
  source 1..122: mol_type = other DNA
gctccgcttctccgggttttagcggaagcctgcggggggcggggtaaccgcggaagccggcggccgtgggcgcgcgggttgggggctctcgcgccgctccgggctctccccccccccggctg

SEQ ID NO: 44 - DNA, 122 bp, synthetic construct
  misc_feature 1..122: note = Description of Artificial Sequence: Synthetic polynucleotide
  source 1..122: mol_type = other DNA
cgcgctccgcttctccgggttttagcggaagcctgcggggggcggggtaaccgcggaagccggcggccgtgggcgcgcgggttgggggctctcgcgccgctccgggctctccccccccccgg

SEQ ID NO: 45 - DNA, 122 bp, synthetic construct
  misc_feature 1..122: note = Description of Artificial Sequence: Synthetic polynucleotide
  source 1..122: mol_type = other DNA
gcgagggatctctgtgcgtcctcactggcccatgcacccagcacctgcgactcccgccgtcgggctgcgtggccccgcgcccacacctgcccgtcccttccgtcgtccctcgctcgcgcaga

SEQ ID NO: 46 - DNA, 122 bp, synthetic construct
  misc_feature 1..122: note = Description of Artificial Sequence: Synthetic polynucleotide
  source 1..122: mol_type = other DNA
ggggaggtgtggggagcggaaggccgcaggagcatctttgcggagaaagtactttggctgcggcgggcgcagggcgggccggctagccccgcgccccacctgttctgtgcgtcgcgctcgcc

SEQ ID NO: 47 - DNA, 122 bp, synthetic construct
  misc_feature 1..122: note = Description of Artificial Sequence: Synthetic polynucleotide
  source 1..122: mol_type = other DNA
tagggctggaaacccgccgccacagcgggctagaggtcgtccccgcccgcaacatatgcgcgaaggaaagtgctacgaacgtcaaatggccgccccccgccgacgccatctgctctgcgaag

SEQ ID NO: 48 - DNA, 122 bp, synthetic construct
  misc_feature 1..122: note = Description of Artificial Sequence: Synthetic polynucleotide
  source 1..122: mol_type = other DNA
cgcccgcaacatatgcgcgaaggaaagtgctacgaacgtcaaatggccgccccccgccgacgccatctgctctgcgaagcagaaacggcggcagctgcgcgcccagtccctccgcccgcgcc

SEQ ID NO: 49 - DNA, 122 bp, synthetic construct
  misc_feature 1..122: note = Description of Artificial Sequence: Synthetic polynucleotide
  source 1..122: mol_type = other DNA
ccccctgttcaaggtctgtcaccgtagggggcgggggggcgcgtggagccgctgggggttcggcccaccccgcgaaccgagctcccggccctgtgcgccctcagctctgccgcgggcgttgg

SEQ ID NO: 50 - DNA, 122 bp, synthetic construct
  misc_feature 1..122: note = Description of Artificial Sequence: Synthetic polynucleotide
  source 1..122: mol_type = other DNA
gctgtggccgcagctgaggcccgacgagcttccggccgggtctttgcccttcactggccgcgtgaacatcacggtgcgctgcacggtggccacctctcgactgctgctgcatagcctcttcc

SEQ ID NO: 51 - DNA, 122 bp, synthetic construct
  misc_feature 1..122: note = Description of Artificial Sequence: Synthetic polynucleotide
  source 1..122: mol_type = other DNA
gtgtgcgtgtgcgtgtgctcagcctcagcgtgaggggcacctgctcgtctgggctcacagcgaaggcagcctcgccgcgagctgccgctgccgctgctgccgccactggtgttgccgctctc

SEQ ID NO: 52 - DNA, 122 bp, synthetic construct
  misc_feature 1..122: note = Description of Artificial Sequence: Synthetic polynucleotide
  source 1..122: mol_type = other DNA
tggggcagcggcgttgcaggagatgagctcagcgcaaagggaaccccgcagcggcgagtgcggctgctggcctgcgcgctgtggccccaacaggctggcagggcgcgggcgggtggcggggt

SEQ ID NO: 53 - DNA, 122 bp, synthetic construct
  misc_feature 1..122: note = Description of Artificial Sequence: Synthetic polynucleotide
  source 1..122: mol_type = other DNA
agagtcggtgggactggctgcgctgccctgaagtggttctccaagcagcgcggagggtggcggacggcggacggagcccaggggccgcgtcgggtggggaaacccgaactcgcggaggggaa

SEQ ID NO: 54 - DNA, 122 bp, synthetic construct
  misc_feature 1..122: note = Description of Artificial Sequence: Synthetic polynucleotide
  source 1..122: mol_type = other DNA
aaagccctggcaggtaaagagaggacccgcgcaggctgggagctcccactcctcctccagcgtcacgctcgccctccgccgctgcctcgcgtccgggtctgtttatatagcgtctggaggcc

SEQ ID NO: 55 - DNA, 122 bp, synthetic construct
  misc_feature 1..122: note = Description of Artificial Sequence: Synthetic polynucleotide
  source 1..122: mol_type = other DNA
ctggccaagtgccggcccatcgcggtgcgcagcggagacgccttccacgagatccggccgcgcgccgaggtggccaacctcagcgcgcacagcgccagccccatccaggatgcggtcctgaa

SEQ ID NO: 56 - DNA, 122 bp, synthetic construct
  misc_feature 1..122: note = Description of Artificial Sequence: Synthetic polynucleotide
  source 1..122: mol_type = other DNA
ggcgccggcagcttcgcgccggcggctggaagcgggcgggctgcacgggcggctcgagtgcggggaccccagcccctcgccctcgtgagcgccgcccctgccacctgctgccaagtcaccgg

SEQ ID NO: 57 - DNA, 122 bp, synthetic construct
  misc_feature 1..122: note = Description of Artificial Sequence: Synthetic polynucleotide
  source 1..122: mol_type = other DNA
ccccggccgcgccgggcgcggggctcgggattcgggagaccgcgcggcgccgaagccacgcgtcagccccactgtcccgcgcgcctcgccccaggcctcgggctcttcctccgcacctcgta

SEQ ID NO: 58 - DNA, 122 bp, synthetic construct
  misc_feature 1..122: note = Description of Artificial Sequence: Synthetic polynucleotide
  source 1..122: mol_type = other DNA
acgcggggactggaaagggcgcctgggtgggaagaggcgctggcgggtgatcgtccccaccgggccagtccccgggatctgctgccgcccctctccgaaattcacagccagagcgggcgcac

SEQ ID NO: 59 - DNA, 122 bp, synthetic construct
  misc_feature 1..122: note = Description of Artificial Sequence: Synthetic polynucleotide
  source 1..122: mol_type = other DNA
tctgagaagtgtcctcctcgctctcttataaaaacaggacttgttgccgaggtcagcgcgcgcatcgagtgtgccaggcgtgtgcgtggtttctgctgtgtcattgctttcacggaaggtgg

SEQ ID NO: 60 - DNA, 122 bp, synthetic construct
  misc_feature 1..122: note = Description of Artificial Sequence: Synthetic polynucleotide
  source 1..122: mol_type = other DNA
gcgcccagactgcgcgccgcgccgctgcgcccaacattcccgaggacggcttcgcgggcgcgtatcgtccagaccggagcaccgccccaccgctagcgcaggagacctgccggggaagtcgc

SEQ ID NO: 61 - DNA, 122 bp, synthetic construct
  misc_feature 1..122: note = Description of Artificial Sequence: Synthetic polynucleotide
  source 1..122: mol_type = other DNA
aaaggccgtactctgccccccgcgggacccaggtccccgcctgctgcagagcgcactctgcgcacgtcgagccgcgaaaggttcacagaagaaaacaagagaaagaagtagcaggcactgag

SEQ ID NO: 62 - DNA, 122 bp, synthetic construct
  misc_feature 1..122: note = Description of Artificial Sequence: Synthetic polynucleotide
  source 1..122: mol_type = other DNA
ggaatccattcttttaagccagggtttaaaactcttcaagcaagtcatctgcaaaggtaccgcttctaccattttaaagataggattatgttccctaggacaactggatgagccctaggaac

SEQ ID NO: 63 - DNA, 122 bp, synthetic construct
  misc_feature 1..122: note = Description of Artificial Sequence: Synthetic polynucleotide
  source 1..122: mol_type = other DNA
gaggagcgcgccgctgcctctggcgggctttcggcttgaggggcaaggtgaagagcgcaccggccgtggggtttaccgagctggatttgtatgttgcaccatgccttcttggatcggggctg

SEQ ID NO: 64 - DNA, 122 bp, synthetic construct
  misc_feature 1..122: note = Description of Artificial Sequence: Synthetic polynucleotide
  source 1..122: mol_type = other DNA
ccctgtgcgtgccgccgcgctgttgctcgcagtgtgctggcgccgagctcggtggacacgcgcgcagtcagagctgcctctcgccctcgctagctgggctcgcagcctcttcctccctccct

SEQ ID NO: 65 - DNA, 122 bp, synthetic construct
  misc_feature 1..122: note = Description of Artificial Sequence: Synthetic polynucleotide
  source 1..122: mol_type = other DNA
ctctttggcaagtggtttgtgcatcaggagaaactttccacctgcgagccgaaccggcgccgagtgcgtgtgtttctgcctttttttgttgtcgttgcctccacccctccccattcttctct

SEQ ID NO: 66 - DNA, 122 bp, synthetic construct
  misc_feature 1..122: note = Description of Artificial Sequence: Synthetic polynucleotide
  source 1..122: mol_type = other DNA
tggctgccagagcgagtgaggggcgcagaggcggcagagagcggagagccccggtgtctccgcgagggcggcggcggccagcagacggcgatcgaggcgcgcgccacggcacggccagcgca

SEQ ID NO: 67 - DNA, 122 bp, synthetic construct
  misc_feature 1..122: note = Description of Artificial Sequence: Synthetic polynucleotide
  source 1..122: mol_type = other DNA
aagcgcgtggagagccgaaaggtgcggtgggcgcagagggcgggctggctgcggggcgaccgcgcgccggggccatgccgcgctccttcctggtggactcgctagtgctgcgcgaggcgggc

SEQ ID NO: 68 - DNA, 122 bp, synthetic construct
  misc_feature 1..122: note = Description of Artificial Sequence: Synthetic polynucleotide
  source 1..122: mol_type = other DNA
ggggtcgccatgaccgagtggcccaggcccgagcgaagcccgcgcgcggtgagtccgccgcggcccatccgtccctccgcccgccagagcgtccatcgggacgcccacccgggagggtctcg

SEQ ID NO: 69 - DNA, 122 bp, synthetic construct
  misc_feature 1..122: note = Description of Artificial Sequence: Synthetic polynucleotide
  source 1..122: mol_type = other DNA
ccgagcgctgcccccgccggcccgcggctgccagccggccctgcccgcgcccgggccccgcgagcggccgcacttcaccttacggaggggagataatgagatcaattagaggcgccgtcacc

SEQ ID NO: 70 - DNA, 122 bp, synthetic construct
  misc_feature 1..122: note = Description of Artificial Sequence: Synthetic polynucleotide
  source 1..122: mol_type = other DNA
ggcaaccctgactcggaccgctcgggagagccccaggagaggccagcgccgcgcagcagccgccccgctgcgcccacctccccggctgctcccggagggctcacaaaggcggtggccgcccg

SEQ ID NO: 71 - DNA, 122 bp, synthetic construct
  misc_feature 1..122: note = Description of Artificial Sequence: Synthetic polynucleotide
  source 1..122: mol_type = other DNA
gcgggcggcagccgcaagcgaggaatccagcgcagggaaagtagccccagtggggcccggcgcgtcagccccactcgcgtggcaaaacttgcgggggcccccgcgtgccgcgcctcagccca

SEQ ID NO: 72 - DNA, 122 bp, synthetic construct
  misc_feature 1..122: note = Description of Artificial Sequence: Synthetic polynucleotide
  source 1..122: mol_type = other DNA
tcctcgccgtcggggtcctcctcctctgccgacgagttgtcactgggcgaggcgtagctgcgctctacgccgcggaggggcggcctcttggaggcggggaccgggtactcccgctgcagccc

SEQ ID NO: 73 - DNA, 122 bp, synthetic construct
  misc_feature 1..122: note = Description of Artificial Sequence: Synthetic polynucleotide
  source 1..122: mol_type = other DNA
gctgctcgcgctccgccgcccgggagatgcttcctcgcgcggcgcagcgctgaggccgtgcgtgcgccccggctgcgctgcgcgctccccacatacacaagctctccatgtgagctgacagg

SEQ ID NO: 74 - DNA, 122 bp, synthetic construct
  misc_feature 1..122: note = Description of Artificial Sequence: Synthetic polynucleotide
  source 1..122: mol_type = other DNA
cttctcttgaaaaggaggagaatcaacactgggctcacaactcatcagagctgagtcatacgtacatcagcaggacctacgtgggaaccaaatagcaaactcaaattgggaaatttgaggaa

SEQ ID NO: 75 - DNA, 122 bp, synthetic construct
  misc_feature 1..122: note = Description of Artificial Sequence: Synthetic polynucleotide
  source 1..122: mol_type = other DNA
ccgagagccccgcctgcaggcggtgtagatacatgtagatactgtagatactgtagataccgccccggcgccgacttgataaacggtttcgcctcttttggaagccgcctgcgtgtccattt

SEQ ID NO: 76 - DNA, 122 bp, synthetic construct
  misc_feature 1..122: note = Description of Artificial Sequence: Synthetic polynucleotide
  source 1..122: mol_type = other DNA
tgaggagtgaggaggcagaaaggaccgagaacaaggggacccggttccatttctggaccccgtccgcaggctgctcgcccgacttggggtcgctctgccccggacgatcaggacagctgcgt

SEQ ID NO: 77 - DNA, 122 bp, synthetic construct
  misc_feature 1..122: note = Description of Artificial Sequence: Synthetic polynucleotide
  source 1..122: mol_type = other DNA
caaatctatatgaaggatcgaattgcattgaactagcaaacacacacacacacacgcacacgcaaaaactgatgaaagctgaacaaggtctgtagtctagtcaacagtactgcactatgtga

SEQ ID NO: 78 - DNA, 122 bp, synthetic construct
  misc_feature 1..122: note = Description of Artificial Sequence: Synthetic polynucleotide
  source 1..122: mol_type = other DNA
acagtctctcgcctcaaagatctccgccattagtggtagccatttaagaaaacagaattacgatgaataatgatttgaagccaaaaagtcaaaatatcttatttcgcaactgtaattgctgg

SEQ ID NO: 79 - DNA, 122 bp, synthetic construct
  misc_feature 1..122: note = Description of Artificial Sequence: Synthetic polynucleotide
  source 1..122: mol_type = other DNA
ccacacaggcctctccctcggtgcggtagcgagggttgcgggcccaaacgcccgcgcccacggaggcgcctgcgacgactagaagcttccacagccatatgggggcaaagacggcccagtag

SEQ ID NO: 80 - DNA, 25 bp, synthetic construct
  misc_feature 1..25: note = Description of Artificial Sequence: Synthetic polynucleotide
  source 1..25: mol_type = other DNA
ggtttttgggtaggaaggatagtag

SEQ ID NO: 81 - DNA, 25 bp, synthetic construct
  misc_feature 1..25: note = Description of Artificial Sequence: Synthetic polynucleotide
  source 1..25: mol_type = other DNA
aaacaaatctaacccctaaaaaaac

SEQ ID NO: 82 - DNA, 20 bp, synthetic construct
  misc_feature 1..20: note = Description of Artificial Sequence: Synthetic polynucleotide
  source 1..20: mol_type = other DNA
caaactaaacacactaaacc

SEQ ID NO: 83 - DNA, 21 bp, synthetic construct
  misc_feature 1..21: note = Description of Artificial Sequence: Synthetic polynucleotide
  source 1..21: mol_type = other DNA
gggtttttagggttggtggta

SEQ ID NO: 84 - DNA, 24 bp, synthetic construct
  misc_feature 1..24: note = Description of Artificial Sequence: Synthetic polynucleotide
  source 1..24: mol_type = other DNA
tcctcataataataaataacaacc

SEQ ID NO: 85 - DNA, 18 bp, synthetic construct
  misc_feature 1..18: note = Description of Artificial Sequence: Synthetic polynucleotide
  source 1..18: mol_type = other DNA
tatgtatgtggtggggtt

SEQ ID NO: 86 - DNA, 45 bp, synthetic construct
  misc_feature 1..45: note = Description of Artificial Sequence: Synthetic polynucleotide
  source 1..45: mol_type = other DNA
aatgatacggcgaccaccgagatctacactctttccctacacgac

SEQ ID NO: 87 - DNA, 52 bp, synthetic construct
  misc_feature 1..52: note = Description of Artificial Sequence: Synthetic polynucleotide
  source 1..52: mol_type = other DNA
caagcagaagacggcatacgagatagtcatcggtgactggagttcagacgtg

SEQ ID NO: 88 - DNA, 59 bp, synthetic construct
  misc_feature 1..59: note = Description of Artificial Sequence: Synthetic polynucleotide
  variation 34..38: note = n is a, c, g, or t
  source 1..59: mol_type = other DNA
acactctttccctacacgacgctcttccgatctnnnnngggtttttagggttggtggta

SEQ ID NO: 89 - DNA, 58 bp, synthetic construct
  misc_feature 1..58: note = Description of Artificial Sequence: Synthetic polynucleotide
  source 1..58: mol_type = other DNA
gtgactggagttcagacgtgtgctcttccgatcttcctcataataataaataacaacc

SEQ ID NO: 90 - DNA, 67 bp, synthetic construct
  misc_feature 1..67: note = Description of Artificial Sequence: Synthetic polynucleotide
  variation 34..38: note = n is a, c, g, or t
  source 1..67: mol_type = other DNA
acactctttccctacacgacgctcttccgatctnnnnnggtaggtttttgggtaggaaggatagtag

SEQ ID NO: 91 - DNA, 59 bp, synthetic construct
  misc_feature 1..59: note = Description of Artificial Sequence: Synthetic polynucleotide
  source 1..59: mol_type = other DNA
gtgactggagttcagacgtgtgctcttccgatctaaacaaatctaacccctaaaaaaac